(19) United States
(12) Patent Application Publication
(10) Pub. No.: US 2023/0091908 A1
(43) Pub. Date: Mar. 23, 2023

(54) FLUORENE DERIVATIVE, METHOD FOR PRODUCING SAME, AND APPLICATION OF SAME

(71) Applicant: Osaka Gas Chemicals Co., Ltd., Osaka (JP)

(72) Inventors: Koji KONISHI, Osaka (JP); Rie YASUDA, Osaka (JP); Shinsuke MIYAUCHI, Osaka (JP); Ibuki MOTOHIRO, Osaka (JP); Mayato MITSUZANE, Osaka (JP); Yuichiro YASUDA, Osaka (JP)

(73) Assignee: Osaka Gas Chemicals Co., Ltd., Osaka (JP)

(21) Appl. No.: 17/788,071

(22) PCT Filed: Dec. 16, 2020
(86) PCT No.: PCT/JP2020/046886

§ 371 Date: Jun. 22, 2022

(30) Foreign Application Priority Data
Dec. 26, 2019 (JP) 2019-236644

(51) Int. Cl. C07C 69/52; C07C 67/08

(52) U.S. Cl. CPC C07C 69/52; C07C 67/08

(57) ABSTRACT

The di(meth)acrylate compound of the present disclosure is represented by the following formula (1):

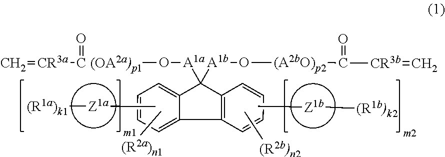

wherein Z^{1a} and Z^{1b} each represent an arene ring, R^{1a} and R^{1b} each represent a substituent, k1 and k2 each denote an integer of not less than 0, m1 and m2 each denote an integer of 0 to 4, and at least one of m1 and m2 denotes 1 or more, R^{2a} and R^{2b} each represent a substituent, n1 and n2 each denote an integer of 0 to 4, m1+n1 and m2+n2 each denote 4 or less, A^{1a} and A^{1b} each represent a straight- or branched-chain alkylene group, A^{2a} and A^{2b} each represent a straight- or branched-chain alkylene group, p1 and p2 each denote an integer of not less than 0, and R^{3a} and R^{3b} each represent a hydrogen atom or a methyl group.

The di(meth)acrylate compound is a novel compound having a high refractive index.

### TECHNICAL FIELD

The present disclosure relates to a novel di(meth)acrylate compound with a fluorene skeleton, a method for producing the same and an application thereof.

### BACKGROUND ART

A compound with a 9,9-bisarylfluorene skeleton has excellent optical properties such as a high refractive index, or other properties, and therefore is effectively used for various optical members as an optical plastic (or an optical resin material). As such a compound with a 9,9-bisarylfluorene skeleton, for example, a number of compounds such as a (meth)acrylate compound have been known. As a (meth)acrylate compound having a particularly high refractive index, Patent Documents 1 and 2 disclose a (meth)acrylate compound with a 9,9-bis condensed polycyclic aryl fluorene skeleton and a curable composition containing the (meth)acrylate compound.

Patent Document 3 discloses a synthesis of 9,9-bis(3-acryloyloxypropyl)fluorene.

### CITATION LIST

### Patent Literature

Patent Document 1: Japanese Patent Application Laid-Open Publication No. 2009-173648 (JP 2009-173648 A)
Patent Document 2: Japanese Patent Application Laid-Open Publication No. 2018-59059 (JP 2018-59059 A)
Patent Document 3: U.S. Patent Application Publication No. 2003/49173 (US 2003/49173 A)

### SUMMARY OF INVENTION

### Technical Problem

In Examples of Patent Documents 1 and 2, a compound having a high refractive index such as 9,9-bis[6-(2-acryloyloxyethoxy)-2-naphthyl]fluorene (BNEFA), a curable composition and a cured product thereof have been prepared.

However, in the field of optical members (components), it is required to obtain the material having even higher refractive index with the expectation further increasing a refractive index of an optical resin material.

Patent Document 3 is a document relating to a quartz crystal microbalance (QCM) sensor element using a molecular imprint polymer (MIP) instead of an optical member and fails to describe any optical characteristics such as a refractive index of 9,9-bis(3-acryloyloxypropyl)fluorene.

It is therefore an object of the present disclosure to provide a novel di(meth)acrylate compound having a high refractive index, a method for producing the same and an application (or use) thereof.

### Solution to Problem

The inventors of the present invention made intensive studies to achieve the above object and finally found the following: regardless of having no a 9,9-bisarylfluorene skeleton, the di(meth)acrylate compound with a specific structure, in which an aryl group is introduced on the 1 to 8-position(s) of a fluorene skeleton thereof, unexpectedly exhibits the above-mentioned extremely high refractive index. The present invention was accomplished based on the above findings.

That is, the di(meth)acrylate compound of the present disclosure is represented by the following formula (1).

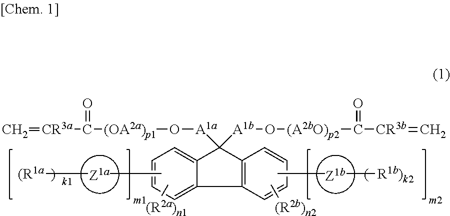

In the formula (1), Z^{1a} and Z^{1b} independently represent an arene ring,

R^{1a} and R^{1b} independently represent a substituent, k1 and k2 independently denote an integer of not less than 0,

m1 and m2 independently denote an integer of 0 to 4, and at least one of m1 and m2 denotes 1 or more,

R^{2a} and R^{2b} independently represent a substituent, n1 and n2 independently denote an integer of 0 to 4,

m1+n1 and m2+n2 each denote 4 or less,

A^{1a} and A^{1b} independently represent a straight- or branched-chain alkylene group,

A^{2a} and A^{2b} independently represent a straight- or branched-chain alkylene group, p1 and p2 independently denote an integer of not less than 0, and

R^{3a} and R^{3b} independently represent a hydrogen atom or a methyl group.

In the formula (1), Z^{1a} and Z^{1b} each may represent a C₆₋₁₂arene ring,

m1 and m2 each may denote an integer of about 1 to 2,

A^{1a} and A^{1b} each may represent a straight- or branched-chain C₁₋₆alkylene group,

A^{2a} and A^{2b} each may represent a straight- or branched-chain C₂₋₄alkylene group, and p1 and p2 each may denote an integer of about 0 to 10.

In the formula (1), Z^{1a} and Z^{1b} each may represent a benzene ring or a naphthalene ring,

m1 and m2 each may denote 1,

A^{1a} and A^{1b} each may represent a straight- or branched-chain C₁₋₄alkylene group, and

p1 and p2 each may denote 0.

The compound may have a refractive index of about 1.65 to 1.75 at a wavelength of 589 nm and a temperature of 20° C.

The present disclosure includes a method for producing the compound. According to this process, the method comprises (or includes) allowing a compound represented by the following formula (2) to react with compounds represented by the following formulae (3a) and (3b).

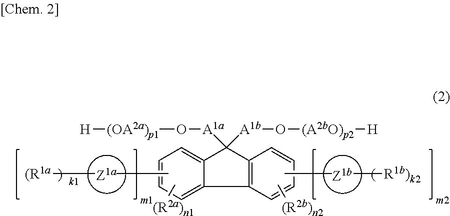

In the formula (2), Z^{1a} and Z^{1b}, R^{1a} and R^{1b}, k1 and k2, m1 and m2, R^{2a} and R^{2b}, n1 and n2, m1+n1 and m2+n2, A^{1a} and A^{1b}, A^{2a} and A^{2b}, and p1 and p2 each have the same meanings as defined in the formula (1).

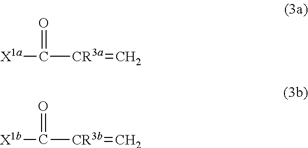

In the formulae (3a) and (3b), X^{1a} and X^{1b} independently represent a hydroxy group, an alkoxy group, or a halogen atom, and R^{3a} and R^{3b} each have the same meanings as defined in the formula (1).

The present disclosure includes a curable composition containing (or comprising) a compound represented by the formula (1). The curable composition may further contain (or comprise) a compound represented by the following formula (7).

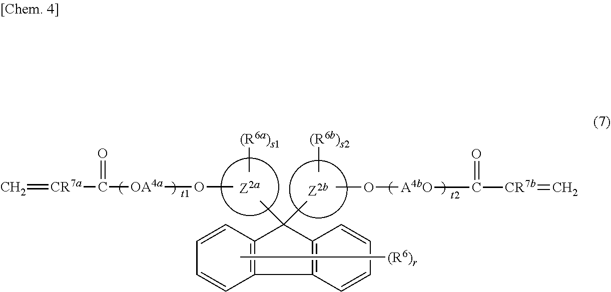

In the formula (7), Z^{2a} and Z^{2b} independently represent an arene ring,

R⁵ represents a substituent, r denotes an integer of 0 to 8,

R^{6a} and R^{6b} independently represent a substituent, s1 and s2 independently denote an integer of not less than 0,

A^{4a} and A^{4b} independently represent a straight- or branched-chain alkylene group, t1 and t2 independently denote an integer of not less than 0, and

R^{7a} and R^{7b} independently represent a hydrogen atom or a methyl group.

In the formula (7), Z^{2a} and Z^{2b} each may represent a C₆₋₁₂arene ring,

R^{6a} and R^{6b} each may represent a hydrocarbon group, s1 and s2 each may denote an integer of about 0 to 2,

A^{4a} and A^{4b} each may represent a straight- or branched-chain C₂₋₄alkylene group, t1 and t2 each may denote an integer of about 0 to 10.

A mass ratio of the compound represented by the formula (1) relative to the compound represented by the formula (7) may be about 10/90 to 90/10 in terms of the former/the latter.

The curable composition may further contain (or comprise) a compound represented by the following formula (8):

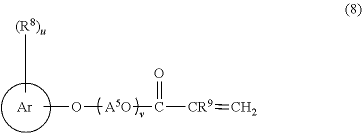

wherein Ar represents an arene ring,

R⁸ represents a substituent, u denotes an integer of not less than 0,

A⁵ represents a straight- or branched-chain alkylene group, v denotes an integer of not less than 0, and

R⁹ represents a hydrogen atom or a methyl group.

In the formula (8), Ar may represent a C₆₋₁₂arene ring,

R⁸ may represent a hydrocarbon group, u may denote an integer of about 0 to 2,

A⁵ may represent a straight- or branched-chain C₂₋₄alkylene group, and v may denote an integer of about 1 to 4.

A mass ratio of the compound represented by the formula (1) relative to the compound represented by the formula (8) may be about 10/90 to 95/5 in terms of the former/the latter.

The present disclosure also includes a cured product in which the curable composition has been cured. The cured product may have:

a refractive index of about 1.65 to 1.75 at a wavelength of 589 nm and a temperature of 20° C.,

a glass transition temperature of about 0 to 50° C., and

a 5% mass reduction temperature of about 330 to 430° C.

Further, the present disclosure includes an optical member containing (or comprising) the cured product.

The present disclosure may solve the following problems as subordinate objects. That is, another object of the present disclosure is to provide a di(meth)acrylate compound capable of forming a cured product which has high heat resistance (high 5% mass reduction temperature) despite having no 9,9-bisarylfluorene skeleton, a method for producing the same and an application (or a use) thereof.

It is still another object of the present disclosure to provide a di(meth)acrylate compound capable of forming a cured product having an excellent flexibility (or toughness) even containing a rigid chemical structure such as an aromatic ring skeleton (or even having a high refractive index and heat resistance), a method for producing the same and an application (or a use) thereof.

It is another object of the present disclosure to provide a di(meth)acrylate compound having an excellent solubility even containing a number of aromatic ring skeletons, a method for producing the same and an application (or a use) thereof.

In this description and claims, the number of carbon atoms in a substituent may be represented as C₁, C₆, C₁₀. For example, an alkyl group having one carbon atom is represented as “C₁alkyl group”, and an aryl group having 6 to 10 carbon atoms is represented as “C₆₋₁₀aryl group”.

### Advantageous Effects of Invention

The novel di(meth)acrylate compound of the present disclosure has an extremely high refractive index despite having no 9,9-bisarylfluorene skeleton. Further, the di(meth)acrylate compound of the present disclosure can form a cured product with relatively high heat resistance (high 5% mass reduction temperature) despite having no 9,9-bisarylfluorene skeleton. A compound having a high refractive index and heat resistance usually contains (has or includes) a rigid chemical structure such as an aromatic ring skeleton (a benzene ring skeleton), tends to have a high glass transition temperature and to form a hard and fragile cured product. In contrast to these facts, unexpectedly, the di(meth)acrylate compound with a specific chemical structure of the present invention easily forms a cured product having a surprisingly lower glass transition temperature and a relatively flexible property (or toughness) even if the compound exhibits the high refractive index and high 5% mass reduction temperature. Furthermore, unexpectedly, the di(meth)acrylate compound easily exhibits a high solubility, even though a compound having a number of aromatic ring skeletons in the chemical structure tends to reduce solubility.

### DESCRIPTION OF EMBODIMENTS

[Di(Meth)Acrylate Compound]

The novel di(meth)acrylate compound of the present disclosure is represented by the following formula (1):

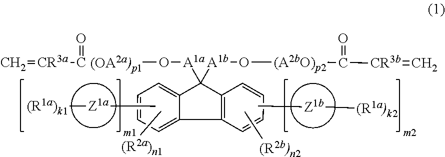

wherein Z^{1a} and Z^{1b} independently represent an arene ring,

R^{1a} and R^{1b} independently represent a substituent, k1 and k2 independently denote an integer of not less than 0,

m1 and m2 independently denote an integer of 0 to 4, and at least one of m1 and m2 denotes 1 or more,

R^{2a} and R^{2b} independently represent a substituent, n1 and n2 independently denote an integer of 0 to 4,

m1+n1 and m2+n2 each denote 4 or less,

A^{1a} and A^{1b} independently represent a straight- or branched-chain alkylene group,

A^{2a} and A^{2b} independently represent a straight- or branched-chain alkylene group, p1 and p2 independently denote an integer of not less than 0, and

R^{3a} and R^{3b} independently represent a hydrogen atom or a methyl group.

In the formula (1), the arene ring (aromatic hydrocarbon ring) represented by Z^{1a} and Z^{1b} may include, for example, a monocyclic arene ring such as a benzene ring, and a polycyclic arene ring. Examples of the polycyclic arene ring may include a condensed polycyclic arene ring (a condensed polycyclic aromatic hydrocarbon ring) and a ring-assemblies arene ring or ring-assembled arene ring (a ring-assemblies polycyclic aromatic hydrocarbon ring).

The condensed (fused) polycyclic arene ring may include, for example, a condensed bi- to tetra-cyclic arene ring such as a condensed bicyclic arene ring and a condensed tricyclic arene ring. Examples of the condensed bicyclic arene ring may include a condensed bicyclic C₁₀₋₁₆arene ring such as a naphthalene ring and an indene ring. The condensed tricyclic arene ring may include, for example, a condensed tricyclic C₁₄₋₂₀arene ring such as an anthracene ring and a phenanthrene ring. A preferred condensed polycyclic arene ring is a condensed polycyclic C₁₀₋₁₄arene ring such as a naphthalene ring.

The ring-assemblies arene ring may include, for example, a biarene ring such as a biphenyl ring, a phenylnaphthalene ring, and a binaphthyl ring; and a terarene ring such as a terphenyl ring. A preferred ring-assemblies arene ring is a biC₁₂₋₁₈arene ring such as a biphenyl ring.

In this description and claims, the term “ring-assemblies (or ring-aggregated) arene ring” means an arene ring which has two or more ring-system (arene ring-system) directly connected by a single bond or a double bond, and has one smaller number of bonds directly connected to each ring-system than the number of the ring-system. As mentioned above, for example, a biarene ring such as a biphenyl ring, a phenylnaphthalene ring, and a binaphthyl ring is classified into a ring-assemblies arene ring even having a condensed polycyclic arene ring skeleton such as a naphthalene ring skeleton. Therefore, the term “ring-assemblies arene ring” is clearly distinguished from the term “condensed polycyclic arene ring” such as a naphthalene ring (non-ring-assemblies arene ring).

Preferred rings Z¹ and Z² each include a C₆-14arene ring, more preferably a C₆₋₁₂arene ring such as a benzene ring, a naphthalene ring and a biphenyl ring, further preferably a C₆₋₁₀arene ring such as a benzene ring and a naphthalene ring, and particularly a naphthalene ring.

The species of the rings Z^{1a} and Z^{1b} may be different from each other, and the same species are preferred. In a case where m1 denotes 2 or more, the species of the two or more rings Z^{1a} may be the same or different from each other. The same relationship between m1 and Z^{1a} also applies to m2 and Z^{1b}

Further, the rings Z^{1a} and Z^{1b} each may be bonded at any one of 1- to 4-positions, and 5- to 8-25 positions of the fluorene skeleton, and preferably at 2-, 3- and/or 7-positions of the fluorene skeleton. In a case where m1 and m2 each denote 1, preferred substitution positions (or bonding positions) of the rings Z^{1a} and Z^{1b} are symmetrical positions on the paper surface in the above formula (1) such as 1,8-positions, 2,7-positions, 3,6-positions, and 4,5-positions, and particularly 2,7-positions.

The substituent represented by R^{1a} and R^{1b} (non-reactive substituent or non-polymerizable substituent) may include, for example, a halogen atom; a hydrocarbon group (or group [—R^{h}]); a group [—OR^{h}] (wherein R^{h} represents the above-mentioned hydrocarbon group); a group [—SR^{h}] (wherein R^{h} represents the above-mentioned hydrocarbon group); an acyl group; a nitro group; a cyano group; and a mono- or di-substituted amino group.

The halogen atom may include, for example, a fluorine atom, a chlorine atom, a bromine atom, and an iodine atom.

The hydrocarbon group represented by the above R^{h} may include, for example, an alkyl group, a cycloalkyl group, an aryl group, and an aralkyl group.

The alkyl group may include, for example, a straight- or branched-chain C₁₋₁₀alkyl group such as methyl group, ethyl group, propyl group, isopropyl group, n-butyl group, isobutyl group, s-butyl group, and t-butyl group, preferably a straight- or branched-chain C₁₋₆alkyl group, and more preferably a straight- or branched-chain C₁₋₄alkyl group.

Examples of the cycloalkyl group may include a C₅₋₁₀cycloalkyl group such as cyclopentyl group and cyclohexyl group.

The aryl group may include, for example, a C₆₋₁₂aryl group such as phenyl group, an alkylphenyl group, biphenylyl group, and naphthyl group. Examples of the alkylphenyl group may include a mono- to tri-C₁₋₄alkyl-phenyl group such as methylphenyl group (or tolyl group) and dimethylphenyl group (or xylyl group).

The aralkyl group may include, for example, a C₆₋₁₀aryl-C₁₋₄alkyl group such as benzyl group and phenethyl group.

The above-mentioned group [—OR^{h}] may include, for example, an alkoxy group, a cycloalkyloxy group, an aryloxy group, and an aralkyloxy group.

Specifically, the group [—OR^{h}] may include a group corresponding to the above-exemplified hydrocarbon group R^{h}. Examples of the alkoxy group may include a straight- or branched-chain C₁₋₁₀alkoxy group such as methoxyl group, ethoxyl group, propoxy group, n-butoxy group, isobutoxy group, and t-butoxy group. Examples of the cycloalkyloxy group may include a C₅-10cycloalkyloxy group such as cyclohexyloxy group. Examples of the aryloxy group may include a C₆₋₁₀aryloxy group such as phenoxy group. Examples of the aralkyloxy group may include a C₆₋₁₀aryl-C₁₋₄alkyloxy group such as benzyloxy group.

The above-mentioned group [—SR^{h}] may include, for example, an alkylthio group, a cycloalkylthio group, an arylthio group, and an aralkylthio group. Specifically, the group [—SR^{h}] may include a group corresponding to the above-exemplified hydrocarbon group R^{h}. Examples of the alkylthio group may be a C₁₋₁₀alkylthio group such as metylthio group, ethylthio group, propylthio group, n-butylthio group, and t-butylthio group. Examples of the cycloalkylthio group may be a C₅₋₁₀cycloalkylthio group such as cyclohexylthio group. Examples of the arylthio group may be a C₆₋₁₀arylthio group such as thiophenoxy group (phenylthio group). Examples of the aralkylthio group may be a C₆₋₁₀aryl-C₁₋₄alkylthio group such as benzylthio group.

Examples of the acyl group may include a C₁₋₆alkyl-carbonyl group such as acetyl group.

The mono- or di-substituted amino group may include, for example, a dialkylamino group and a bis(alkylcarbonyl)amino group. Examples of the dialkylamino group may include a diC₁₋₄alkylamino group such as dimethylamino group. Examples of the bis(alkylcarbonyl)amino group may include a bis(C₁₋₄alkyl-carbonyl)amino group such as diacetylamino group.

Representative examples of these groups R^{1a} and R^{1b} may include the hydrocarbon group, the alkoxy group, the acyl group, the nitro group, the cyano group, and the substituted amino group. In a case where k1 denotes not less than 1, the group R^{1a} is preferably the alkyl group and the alkoxy group, specifically a straight- or branched-chain C₁₋₆alkyl group such as methyl group, and a straight- or branched-chain C₁₋₄alkoxy group such as methoxy group. Among these substituents, the alkyl group is preferred, and particularly, a straight- or branched-chain C₁₋₄alkyl group such as methyl group. The same relationship between k1 and R^{1a} also applies to k2 and R^{1b}. In a case where the group R^{1a} is the aryl group, the group R^{1a} may form the ring-assemblies arene ring together with the ring Z^{1a}. The same relationship between R^{1a} and Z^{1a} also applies to R^{1b} and Z^{1b}.

The numbers k1 and k2 of the substituents may suitably be selected according to the species of the rings Z^{1a} and Z^{1b}. The numbers k1 and k2 may be, for example, selected from a range of an integer of about 0 to 7, and a preferred range of the numbers k1 and k2 is an integer of 0 to 6, an integer of 0 to 5, an integer of 0 to 4, an integer of 0 to 3, and an integer of 0 to 2 in a stepwise manner. More preferred k1 and k2 each denote 0 or 1, particularly 0.

The number k1 of substituents R^{1a} may be different from the number k2 of substituents R^{1b}, and the same numbers are preferred. In a case where the number k1 of substituents denotes 2 or more, the species of the two or more groups R^{1a} bonded to the same ring Z^{1a} may be the same or different from each other. The same relationship between k1 and R^{1a} also applies to k2 and R^{1b}. Further, the species of the groups R^{1a} and R^{1b} may be different from each other, and the same species are preferred. The substitution positions of the groups R^{1a} and R^{1b} are not particularly limited to specific positions, and may be selected depending on the species of the rings Z^{1a} and Z^{1b}.

The numbers m1 and m2 of the substituents [—Z^{1a}—(R^{1a})ₖ₁] and [—Z^{1b}—(R^{1b})ₖ₂] (hereinafter, these substituents each may be also referred to as a Z¹-containing group) each denote, for example, an integer of about 1 to 3, preferably 1 or 2, and more preferably 1. The numbers m1 and m2 of the substituents may be different from each other, and the same numbers are preferred. Of these numbers m1 and m2, at least one of m1 and m2 denotes an integer of 1 or more, preferably both denote an integer of 1 or more such as 1 to 2, and more preferably both denote 1.

In a case where the number m1 of substituents denotes 2 or more, the species of the two or more groups [—Z^{1a}—(R^{1a})ₖ₁] may be the same or different from each other. The same relationship between m1 and group [—Z^{1a}—(R^{1a})ₖ₁] also applies to m2 and group [—Z^{1b}_(R^{1b})ₖ₂]. Further, in a case where both of the numbers m1 and m2 of substituents denote 1 or more, the species of the groups [—Z^{1a}—(R^{1a})ₖ₁] and [—Z^{1b}—(R^{1b})ₖ₂] may be the same or different from each other, and the same species are preferred.

The substituent represented by R^{2a} and R^{2b} (non-reactive substituent or non-polymerizable substituent) is a substituent other than the above-mentioned Z¹-containing group. Representative examples of these groups R^{2a} and R^{2b} may include a hydrocarbon group such as an alkyl group (provided that an aryl group is excluded); a halogen atom such as a fluorine atom, chlorine atom, and bromine atom; and a cyano group. Examples of the alkyl group may include a straight- or branched-chain C₁₋₆alkyl group such as methyl group, ethyl group, and t-butyl group. In a case where the number n1 of substituent (s) denotes not less than 1, the group R^{2a} is preferably a straight- or branched-chain C₁₋₄alkyl group, and more preferably a straight- or branched-chain C₁₋₃alkyl group. Among these alkyl groups, a C₁₋₂alkyl group such as methyl group is particularly preferred. The same relationship between n1 and R^{2a} also applies to n2 and R^{2b}.

The numbers n1 and n2 of the substituents R^{2a} and R^{2b} each denote, for example, an integer of about 0 to 3, preferably an integer of 0 to 2, more preferably 0 or 1, and particularly 0. The numbers n1 and n2 of the substituents may be different from each other, and the same numbers are preferred. In a case where the number n1 denotes 2 or more, the species of plural groups R^{2a} may be the same or different from each other. The same relationship between n1 and R^{2a} also applies to n2 and R^{2b}. Further, in a case where both of the numbers n1 and n2 of substituents denote 1 or more, the species of the groups R^{2a} and R^{2b} may be the same or different from each other, and the same species are preferred. The substitution positions of the groups R^{2a} and R^{2b} are not particularly limited to specific positions, and the groups R^{2a} and R^{2b} are bonded to a position other than the substitution position of the Z¹-containing group.

The total numbers m1+m2 and n1+n2 each denote, for example, an integer of 0 to 4, preferably an integer of 1 to 3, more preferably 1 or 2, and further preferably 1. The total numbers m1+m2 and n1+n2 may be different from each other, and the same numbers are preferred.

The straight- or branched-chain alkylene group represented by A^{1a} and A^{1b} may include, for example, a straight- or branched-chain C₁₋₁₂alkylene group such as methylene group, ethylene group, trimethylene group, propylene group, 1,2-butanediyl group, and 2-methylpropane-1,3-diyl group. The alkylene group is preferably a straight- or branched-chain C₁₋₈alkylene group, specifically a straight- or branched-chain C₁-6alkylene group such as methylene group, ethylene group, trimethylene group, propylene group, and 2-methylpropane-1,3-diyl group, more preferably a straight- or branched-chain C₁₋₅alkylene group, and further preferably a straight- or branched-chain C₁₋₄alkylene group. Among these alkylene group, a straight- or branched-chain C₂₋₄alkylene group is preferred, and particularly trimethylene group. The species of the groups A^{1a} and A^{1b} may be different from each other, and the same species are preferred.

The straight- or branched-chain alkylene group represented by A^{2a} and A^{2b} may include, for example, a straight- or branched-chain C₂₋₆alkylene group such as ethylene group, propylene group, trimethylene group, 1,2-butanediyl group, 1,3-butanediyl group, and tetramethylene group. The alkylene group is preferably a straight- or branched-chain C₂₋₄alkylene group, and more preferably a straight- or branched-chain C₂₋₃alkylene group. Among these alkylene group, ethylene group and propylene group are preferred, and ethylene group is particularly preferred. The species of the groups A^{2a} and A^{2b} may be different from each other, and the same species are preferred.

The repeating numbers p1 and p2 of oxyalkylene groups (OA^{2a}) and (OA^{2b}) each may, for example, be selected from a range of about 0 to 20. A preferred range of the repeating numbers p1 and p2 is 0 to 15, 0 to 10, 0 to 8, 0 to 5, 0 to 3, 0 to 2, and 0 to 1 in a stepwise manner. In particular, preferred repeating numbers p1 and p2 each are 0. In a case where p1 denotes 2 or more, the species of the two or more groups A^{2a} in a (poly)oxyalkylene group [-(OA^{2a})ₚ₁-] may be different from each other, and the same species are preferred. The same relationship between p1 and A^{2a} also applies to p2 and A^{2b}.

The numbers of p1 and p2 may be the same or different from each other. The repeating numbers p1 and p2 each may be an average (arithmetic average, arithmetical average), that is, an average addition molar amount; and the range of the average, including preferred embodiments (ranges), is within the above-mentioned integer range.

The total number of the repeating numbers p1 and p2 means a total number of oxyalkylene groups (OA^{2a}) and (OA^{2b}) per molecule of the di(meth)acrylate compound represented by the formula (1) (or an average of total addition molar amounts), and may simply be referred to as p1+p2. The total number p1+p2 may for example be selected from a range of about 0 to 30. A preferred range of p1+p2 is 0 to 25, 0 to 20, 0 to 15, 0 to 12, 0 to 10, 0 to 8, 0 to 6, 0 to 5, 0 to 4, 0 to 3, and 0 to 2 in a stepwise manner. More preferred p1+p2 denotes 0 to 1, and particularly 0. The total number p1+p2 may an integer as mentioned above, and may be the above-mentioned average of total addition molar amounts. The range of the average of total addition molar amounts, including preferred embodiments (ranges), is within the above-mentioned integer range.

An excessively large repeating number p1, p2, or p1+p2 may lead to decrease in refractive index and heat resistance.

The total number p1+p2 can be measured by conventional methods. For example, a compound represented by the formula (2) described later (diol compound), which is a raw material of a di(meth)acrylate compound represented by the formula (1), may be prepared by addition-reacting a compound represented by the formula (4) described later (diol compound) with an alkylene oxide (an alkylene carbonate or haloalkanol) to form (poly)alkyleneoxy groups [-(OA^{2a})ₚ₁-] and [-(OA^{2b})ₚ₂-]. The total number p1+p2 may be measured or evaluated as an arithmetic average or arithmetical average based on the amount ratio of the consumed alkylene oxide (the alkylene carbonate or haloalkanol) in the reaction relative to the diol compound (or hydroxy value). Specifically, the total number p1+p2 can be measured by the method described in Japanese Patent Application Laid-Open Publication No. 2013-53310 (JP 2013-53310 A) or other methods.

The groups R^{3a} and R^{3b} each may represent either a hydrogen atom or a methyl group, and preferably a hydrogen atom from a viewpoint of improving or increasing reactivity (or curability) and refractive index. The species of the groups R^{3a} and R^{3b} may be the same or different from each other, and the same species are preferred.

Representative examples of a di(meth)acrylate compound represented by the formula (1) may include, for example, a di(meth)acrylate compound in which each of m1 and m2 denotes 1, and each of p1 and p2 denotes 0, that is, a 9,9-bis[(meth)acryloyloxyalkyl]-diarylfluorene. More specifically, the di(meth)acrylate compound may include a 9,9-bis[(meth)acryloyloxyalkyl]-diphenylfluorene and a 9,9-bis[(meth)acryloyloxyalkyl]-dinaphthyl fluorene.

The 9,9-bis[(meth)acryloyloxyalkyl]-diphenylfluorene may include, for example, a 9,9-bis[(meth)acryloyloxyC₁₋₆alkyl]-diphenylfluorene such as 9,9-bis[3-(meth)acryloyloxypropyl]-1,8-diphenylfluorene, 9,9-bis[3-(meth)acryloyloxypropyl]-2,7-diphenylfluorene, 9,9-bis[3-(meth)acryloyloxypropyl]-3,6-diphenylfluorene, and 9,9-bis[3-(meth)acryloyloxypropyl]-4,5-diphenylfluorene.

Examples of the 9,9-bis[(meth)acryloyloxyalkyl]-dinaphthyl fluorene may include a 9,9-bis[(meth)acryloyloxyC₁₋₆alkyl]-dinaphthyl fluorene such as 9,9-bis[3-(meth)acryloyloxypropyl]-1,8-di(2-naphthyl)fluorene, 9,9-bis[3-(meth)acryloyloxypropyl]-2,7-di(2-naphthyl)fluorene, 9,9-bis[3-(meth)acryloyloxypropyl]-3,6-di(2-naphthyl)fluorene, 9,9-bis[3-(meth)acryloyloxypropyl]-4,5-di(2-naphthyl)fluorene, and 9,9-bis[3-(meth)acryloyloxypropyl]-2,7-di(1-naphthyl)fluorene.

Among these di(meth)acrylate compounds represented by the formula (1), a 9,9-bis[(meth)acryloyloxyC₁₋₄alkyl]-2,7-diphenylfluorene such as 9,9-bis[3-(meth)acryloyloxypropyl]-2,7-diphenylfluorene; and a 9,9-bis[(meth)acryloyloxypropylC₁₋₄alkyl]-2,7-dinaphthylfluorene are preferred, and more preferably a 9,9-bis[(meth)acryloyloxyC₂₋₄alkyl]-2,7-di(2-naphthyl)fluorene, and particularly a 9,9-bis[(meth)acryloyloxyC₂₋₃alkyl]-2,7-di(2-naphthyl)fluorene such as 9,9-bis[3-(meth)acryloyloxypropyl]-2,7-di(2-naphthyl)fluorene.

The di(meth)acrylate compound represented by the formula (1) has a high refractive index. The di(meth)acrylate compound may have a refractive index nD (refractive index before curing) of about 1.6 to 1.8 at a temperature of 25° C. and a wavelength of 589 nm; and a preferred range of the refractive index is 1.63 to 1.77, 1.65 to 1.75, 1.655 to 1.72, 1.66 to 1.7, 1.665 to 1.695, 1.67 to 1.69, and 1.675 to 1.685 in a stepwise manner.

The di(meth)acrylate compound represented by the formula (1) may have, for example, a melting point of about 50 to 200° C.; and the melting point is preferably 80 to 160° C., 100 to 140° C., 110 to 130° C., and 115 to 125° C. in a stepwise manner.

The di(meth)acrylate compound represented by the formula (1) tends to have an excellent solubility even having a number of aromatic ring skeletons (benzene ring skeletons), which is liable to reduce a solubility, in the chemical structure. For example, the di(meth)acrylate compound is easily soluble in various species of solvents compared to conventional polyfunctional (meth)acrylates with the same number of aromatic ring skeletons (benzene ring skeletons) (e.g., polyfunctional (meth)acrylate described in Japanese Patent Application Laid-Open Publication No. 2018-59059 (JP 2018-59059 A), even at a relatively high concentration of about 20 to 5% by mass, preferably 25 to 40% by mass, and more preferably 30 to 35% by mass. Therefore, the compound represented by the formula (1) is compatible high refractive index and/or high heat resistance with high solubility, and even if the compound is a solid having no fluidity at room temperature of about 25° C., the handleability thereof can be effectively improved.

A melt viscosity of the di(meth)acrylate compound represented by the formula (1) at 150° C. may be, for example, about 10 to 1000 mPa-s; and the viscosity is preferably 50 to 500 mPa-s, 100 to 400 mPa-s, 150 to 350 mPa-s, and 200 to 300 mPa-s in a stepwise manner.

In this description and claims, the refractive index, the melting point and the melt viscosity of the di(meth)acrylate compound represented by the formula (1) can be measured according to the methods described in Examples mentioned below.

[Production Method of Di(Meth)Acrylate Compound]

The method for producing the di(meth)acrylate compound represented by the formula (1) is not particularly limited to a specific one. For example, the di(meth)acrylate compound may be prepared by the following reaction steps:

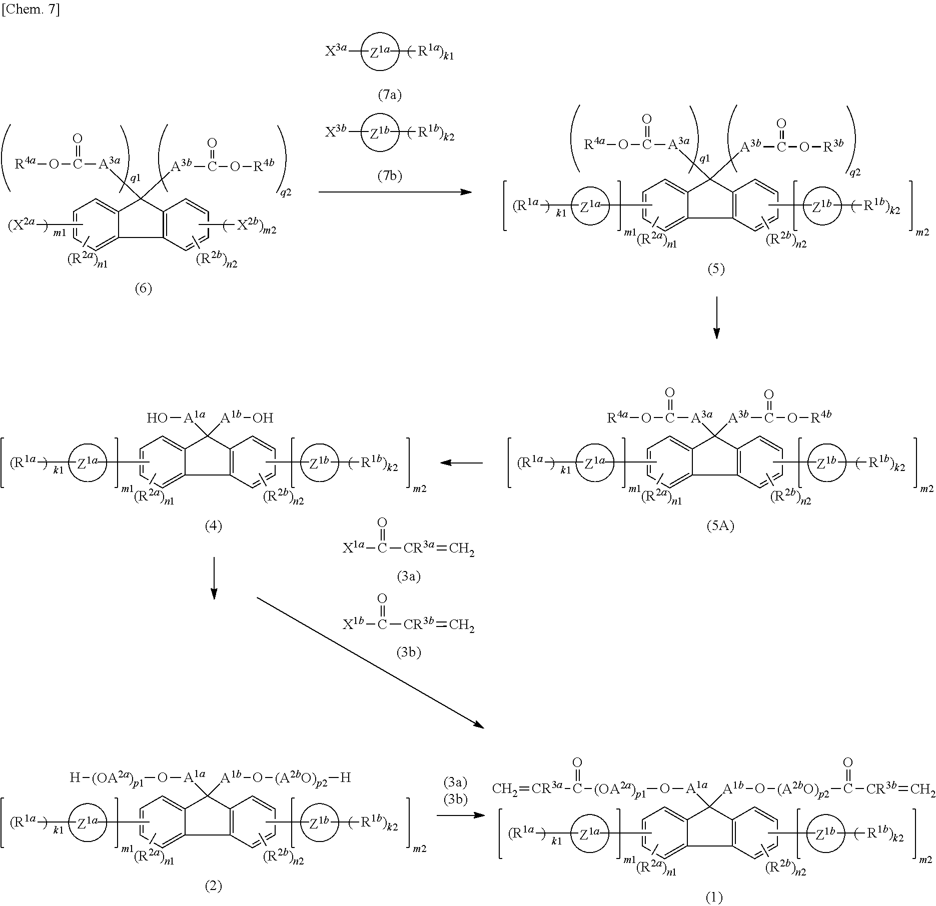

wherein X^{1a} and X^{1b} independently represent a hydroxy group, an alkoxy group, or a halogen atom,

R^{4a} and R^{4b} independently represent a hydrogen atom or an alkyl group,

A^{3a} and A^{3b} independently represent a straight- or branched-chain alkylene group,

q1 and q2 independently denote 0 or 1,

X^{2a} and X^{2b} independently represent a reactive group capable of forming carbon-carbon bond (or direct bond) by coupling reaction; X^{3a} and X^{3b} each represent a reactive group capable of forming carbon-carbon bond by coupling reaction with the reactive groups X^{2a} and X^{2b}, respectively, and Z^{1a} and Z^{1b}, R^{1a} and R^{1b}, k1 and k2, m1 and m2, R^{2a} and R^{2b}, n1 and n2, m1+n1 and m2+n2, A^{1a} and A^{1b}, A^{2a} and A^{2b}, p1 and p2, and R^{3a} and R^{3b}, including preferred embodiments, each have the same meanings as defined in the formula (1).

(Preparation of Compound Represented by Formula (5))

A compound represented by the formula (5) can be prepared by a coupling reaction (or cross-coupling reaction) between a compound represented by the formula (6) and compounds represented by the formulae (7a) and (7b).

The coupling reaction is not particularly limited to a specific one, and may be conventional one, for example, a palladium-catalyzed (or palladium(0)-catalyzed) coupling reaction such as Suzuki-Miyaura coupling reaction, Migita-Kosugi-Stille coupling reaction, Negishi coupling reaction, and Hiyama coupling reaction; and a nickel-catalyzed (or nickel(0)-catalyzed) coupling reaction such as Kumada-Tamao-Corriu coupling reaction. Among these coupling reactions, Suzuki-Miyaura coupling reaction is preferred.

The reactive groups X^{2a} and X^{2b}, and X^{3a} and X^{3b} can be appropriately selected depending on the nature (or kind or type) of the coupling reaction. In a case where Suzuki-Miyaura coupling reaction is used for synthesizing the compound represented by the formula (5), one reactive group (or a first reactive group), for example, each of the groups X^{2a} and X^{2b} may include a halogen atom or a fluoroalkanesulfonyloxy group. Examples of the halogen atom may include an iodine atom, a bromine atom, and a chlorine atom. Examples of the fluoroalkanesulfonyloxy group may be a fluoroC₁₋₄alkanesulfonyloxy group such as trifluoromethanesulfonyloxy group (or group [-OTf]).

These one reactive groups may be used alone or in combination of two or more. Among these one reactive groups, a halogen atom is preferred, more preferably an iodine atom and a bromine atom, and particularly a bromine atom.

In Suzuki-Miyaura coupling reaction, one reactive group mentioned above (or the first reactive group) is capable of coupling with the other reactive group (or a second reactive group); and the other reactive group (or the second reactive group), for example, each of the groups X^{3a} and X^{3b} may include, for example, a boronic acid group (a dihydroxyboryl group or a group [—B(OH)₂]), and a boronic acid ester (borate) group. The boronic acid ester (borate) group may include, for example, a dialkoxyboryl group such as dimethoxyboryl group, diisopropoxyboryl group, and dibutoxyboryl group; and a cyclic boronic acid ester (borate) group such as pinacolatoboryl group (or a group [-Bpin]), 1,3,2-dioxabolinan-2-yl group (1,3,2-dioxaborolan-2-yl group), and 5,5-dimethyl-1,3,2-dioxabolinan-2-yl group (5,5-dimethyl-1,3,2-dioxaborolan-2-yl group).

These other reactive groups mentioned above may be used alone or in combination of two or more. Among the other reactive groups, a group [—B(OH)₂] is preferred,

As long as the groups X^{2a} and X^{2b}, and the groups X^{3a} and X^{3b} form a pair of reactive groups capable of coupling with each other, respectively, these groups X^{2a} and X^{2b}, and X^{3a} and X^{3b} may be any reactive group. The groups X^{2a} and X^{2b} may be the second reactive group such as a boronic acid group, and the groups X^{3a} and X^{3b} may be the first reactive group such as a halogen atom. The groups X^{2a} and X^{2b} may preferably be the first reactive group such as a halogen atom, and the groups X^{3a} and X^{3b} may preferably be the second reactive group such as a boronic acid group.

In the formula (6), the substitution positions of the groups X^{2a} and X^{2b}, including preferred embodiments (substitution positions), correspond to those of the Z¹-containing groups in the formula (1), and are the same as described above.

In the formula (6) [and formulae (5) and (5A)], the straight- or branched-chain alkylene group represented by each of each of A^{3a} and A^{3b} corresponds to an alkylene group represented by A^{1a} and A^{1b}, and has one less carbon atoms than the alkylene group represented by each of A^{1a} and A^{1b}. Representative examples of the alkylene groups A^{3a} and A^{3b} may be a straight- or branched-chain C₁₋₁₁alkylene group such as methylene group, ethylene group, trimethylene group, propylene group, 1,2-butanediyl group, and 2-methylpropane-1,3-diyl group. The alkylene groups A^{3a} and A^{3b} are preferably a straight- or branched-chain C₁₋₅alkylene group, and more preferably a straight- or branched-chain C₁₋₄alkylene group, further preferably a straight- or branched-chain C₁₋₃alkylene group, and particularly ethylene group. In a case where both of the numbers q1 and q2 denote 1, the species of the groups A^{3a} and A^{3b} may be different from each other, and the same species are preferred.

In the formula (6) [and formulae (5) and (5A)], the alkyl group represented by R^{4a} and R^{4b} may include, for example, a straight- or branched-chain C₁₋₆alkyl group such as methyl group, ethyl group, propyl group, isopropyl group, n-butyl group, and t-butyl group. The alkyl groups R^{4a} and R^{4b} are preferably a straight- or branched-chain C₁₋₄alkyl group, more preferably a straight- or branched-chain C₁₋₃alkyl group, and particularly a C₁₋₂alkyl group such as methyl group.

The groups R^{4a} and R^{4b} each may represent either a hydrogen atom or an alkyl group, and preferably an alkyl group. In a case where both q1 and q2 denote 1, the species of the groups R^{4a} and R^{4b} may be different from each other, and the same species are preferred.

In the formula (6) [and formula (5)], the coefficient q1 of a group [-A³a-C(═O)—O—R^{4a}] and the coefficient q2 of a group [-A^{3b}-C(═O)—O—R^{4b}] each may denote 0 or 1, and preferably denote 1. Further the numbers of q1 and q2 may be different numbers from each other, and the same numbers are preferred.

Representative compounds represented by the formula (6) may include, for example, a dihalo-9H-fluorene and a 9,9-bis(alkoxycarbonylalkyl)dihalofluorene.

The dihalo-9H-fluorene may include, for example, 2,7-dibromo-9H-fluorene. The dihalo-9H-fluorene may be a commercially available product.

The 9,9-bis(alkoxycarbonylalkyl)dihalofluorene may include, for example, a 9,9-bis(C₁₋₄alkoxy-carbonyl-C₂₋₆alkyl)-dihalofluorene such as 9,9-bis(2-methoxycarbonylethyl)-2,7-dibromofluorene, 9,9-bis(2-ethoxycarbonylethyl)-2,7-dibromofluorene, and 9,9-bis(2-methoxycarbonylpropyl)-2,7-dibromofluorene. The 9,9-bis(alkoxycarbonylalkyl)dihalofluorene may be prepared, for example, according to the method described in Japanese Patent Application Laid-Open Publication No. 2005-89422 (JP 2005-89422 A). Specifically, the 9,9-bis(alkoxycarbonylalkyl)dihalofluorene may be prepared by a method of reacting a 9H-fluorene compound, in which the 9-position is unsubstituted, such as 2,7-dibromofluorene with an acrylic acid ester such as methyl acrylate, or a haloacetic acid ester such as methyl bromoacetate in the presence of a base catalyst such as trimethylbenzylammonium hydroxide.

Among these compounds represented by the formula (6), the 9,9-bis(alkoxycarbonylalkyl)dihalofluorene is preferred.

The compounds represented by the formulae (7a) and (7b) may include a compound corresponding to preferred embodiments of Z^{1a} and Z^{1b}, R^{1a} and R^{1b}, and k1 and k2 in the di(meth)acrylate represented by the formula (1); for example, an arylboronic acid such as phenylboronic acid, 1-naphthylboronic acid, and 2-naphthylboronic acid. Among these compounds, 2-naphthylboronic acid is preferred. The compounds represented by the formulae (7a) and (7b) are preferably the same compounds. The compounds represented by the formulae (7a) and (7b) may be a commercially available product.

The molar ratio of the compound represented by the formula (6) relative to the total amount of the compounds represented by the formulae (7a) and (7b) may for example be about 1/2 to 1/10 in terms of the former/the latter; and a preferred range of the molar ratio is 1/2.2 to 1/8, 1/2.5 to 1/5, and 1/2.7 to 1/3.3 in terms of the former/the latter in a stepwise manner.

In a case of synthesis by Suzuki-Miyaura coupling reaction, the reaction is carried out in the presence of a palladium catalyst. The palladium catalyst may include a conventional coupling catalyst such as a palladium(0) catalyst and a palladium(II) catalyst.

Examples of the palladium(0) catalyst may be a palladium(0)-phosphine complex such as tetrakis(triphenylphosphine)palladium(0) [or Pd(PPh₃)₄], and bis(tri-t-butylphosphine)palladium(0) [or Pd(P(t-Bu)₃)₂].

Examples of the palladium(II) catalyst may be a palladium(II)-phosphine complex such as [1,2-bis(diphenylphosphino)ethane]palladium(II) dichloride [or PdCl₂(dppe)], [1,3-bis(diphenylphosphino)propane]palladium(II) dichloride [or PdCl₂(dppp)], [1,1′-bis(diphenylphosphino)ferrocene]palladium(II) dichloride [or PdCl₂(dppf)], bis(triphenylphosphine)palladium(II) dichloride [or PdCl₂(PPh₃)₂], and bis(tri-o-tolylphosphine)palladium(II) dichloride [or PdCl₂(P(o-Tol)₃)₂]. In a case where the palladium(II) catalyst is used, the reaction is started after reducing the catalyst to a zero-valent (0-valent) complex by a reducing compound in the reaction system. The reducing compound may include, for example, a phosphine, an amine, and an organometallic reagent.

The palladium catalyst may be prepared in a reaction system by adding a catalyst precursor such as tris(dibenzylideneacetone)dipalladium(0) chloroform complex [or Pd₂(dba)₃-CHCl₃], and an ligand such as a phosphine compound and a carbene compound.

These catalysts may be used alone or in combination of two or more. Among these catalysts, a palladium(0)-phosphine complex such as Pd(PPh₃)₄ is preferred. The ratio of the catalysts may for example be about 0.01 to 0.1 mol, and the ratio is preferably 0.03 to 0.07 mol in terms of metal relative to 1 mol of the compound represented by the formula (6).

In Suzuki-Miyaura coupling reaction, the reaction may be carried out in the presence of a base. The base may include, for example, a metal carbonate or bicarbonate (hydrogen carbonate); a metal hydroxide, a metal fluoride, a metal phosphate, a metal organic acid salt, and a metal alkoxide.

Examples of the metal carbonate or bicarbonate may include an alkali metal carbonate or bicarbonate such as sodium carbonate, potassium carbonate, cesium carbonate, and sodium bicarbonate (sodium hydrogen carbonate); and thallium(I) carbonate.

Examples of the metal hydroxide may include an alkali metal hydroxide such as sodium hydroxide, potassium hydroxide, and cesium hydroxide; an alkaline earth metal hydroxide such as barium hydroxide; and thallium(I) hydroxide.

Examples of the metal fluoride may include an alkali metal fluoride such as potassium fluoride and cesium fluoride.

Examples of the metal phosphate may include an alkali metal phosphate such as tripotassium phosphate.

Examples of the metal organic acid salt may include an alkali metal acetate such as potassium acetate.

Examples of the metal alkoxide may include an alkali metal alkoxide such as sodium methoxide, sodium ethoxide, and potassium t-butoxide.

These bases may be used alone or in combination of two or more. Among these bases, a metal carbonate such as potassium carbonate is preferred. The ratio of the bases may for example be about 0.1 to 50 mol, and the ratio is preferably 1 to 25 mol relative to 1 mol of the compound represented by the formula (6).

The coupling reaction may be carried out in the presence or absence of phase transfer catalyst(s). The phase transfer catalyst may include, for example, a tetraalkylammonium halide such as tetrabutylammonium bromide (TBAB), and trioctylmethylammonium chloride. These phase transfer catalysts may be used alone or in combination of two or more. Among these phase transfer catalysts, TBAB is preferred.

The coupling reaction may be carried out in the absence or presence of an inert or inactive solvent to the reaction. Examples of the solvent may include water; an alcohol such as methanol and ethanol; an ether such as a cyclic ether, and a chain ether; a ketone such as acetone and methyl ethyl ketone; an ester such as ethyl acetate; a nitrile such as acetonitrile and benzonitrile; an amide such as N,N-dimethylformamide, dimethylacetamide and N-methyl-2-pyrrolidone; a sulfoxide such as dimethyl sulfoxide; and a hydrocarbon such as an aliphatic hydrocarbon, an alicyclic hydrocarbon, and an aromatic hydrocarbon.

Examples of the cyclic ether may include dioxane and tetrahydrofuran. Examples of the chain ether may include a dialkyl ether such as diethyl ether and diisopropyl ether; and a glycol ether. The glycol ether may include, for example, a (poly)alkylene glycol monoalkyl ether such as methyl cellosolve and methyl carbitol; and a (poly)alkylene glycol dialkyl ether such as dimethoxyethane.

Examples of the aliphatic hydrocarbon may include hexane and dodecane. The alicyclic hydrocarbon may include, for example, cyclohexane. The aromatic hydrocarbon may include, for example, toluene and xylene.

These solvents may be used alone or in combination. Among these solvents, a preferred solvent is a mixed solvent of water and a chain ether such as dimethoxyethane.

The coupling reaction may be carried out in an atmosphere of an inert gas, for example, a nitrogen gas; and a rare gas such as helium and argon. The reaction temperature is, for example, 50 to 200° C., and preferably 60 to 100° C. The reaction time is not particularly limited to a specific time, and may be, for example, about 1 to 10 hours.

After the completion of the reaction, if necessary, the reaction mixture may be separated and purified by a conventional separation and purification means, for example, a method such as washing, extraction, filtration, dehydration, concentration, decantation, recrystallization, reprecipitation, chromatography, and a combination of these methods.

(Compound Represented by Formula (5A))

The compound represented by the formula (5A) corresponds to a compound represented by the formula (5) in which q1 and q2 each denote 1, and can be prepared by using a compound represented by the formula (6) in which q1 and q2 each denote 1, as a raw material. The compound represented by the formula (5A) may, for example, be prepared by reacting a (meth)acrylic ester, a haloacetic acid alkyl ester, or others with the compound represented by the formula (5) in which at least one of q1 and q2 denotes 0, and particularly q1 and q2 each denote 0, according to the method described in Japanese Patent Application Laid-Open Publication No. 2005-89422 (JP 2005-89422 A).

(Preparation of Compound Represented by Formula (4))

The compound represented by the formula (4) can be prepared by reducing the compound represented by the formula (5A). For the reduction, a conventional reducing agent may be used. Examples of the reducing agent may include a metal hydride compound such as a metal borohydride compound, a metal aluminum hydride compound, a borane compound, an aluminum hydride compound, an organosilicon compound, and an organotin compound.

The metal borohydride compound may include, for example, an alkali metal borohydride compound; and a zinc borohydride compound such as zinc borohydride (Zn(BH₄)₂). The alkali metal borohydride compound may include, for example, a lithium borohydride compound such as lithium borohydride (lithium tetrahydroborate) (LiBH₄), lithium triethyl borohydride (LiBH(C₂H₅)₃), lithium tri-s-butyl borohydride (LiBH(s-C₄H₉)₃), and lithium bis(2,4,6-trimethylphenyl) borohydride (LiBH(Mes)₂); a sodium borohydride compound such as sodium borohydride (sodium tetrahydroborate) (NaBH₄), sodium cyanoborohydride (NaBH₃CN), sodium trimethoxyborohydride (NaBH(OCH₃)₃), sodium triacetoxyborohydride (NaBH(OCOCH₃)₃), and sulfide of sodium borohydride (NaBH₂S₃); and a potassium borohydride compound such as potassium tri-s-butyl borohydride (KBH(s-C₄H₉)₃).

Examples of the metal aluminum hydride compound may include an alkali metal aluminum hydride compound. The alkali metal aluminum hydride compound may include, for example, a lithium aluminum hydride compound such as lithium aluminum hydride (LiAlH₄), lithium trimethoxyaluminum hydride (LiAlH(OCH₃)₃), and lithium trit-butoxyaluminum hydride (LiAlH(Ot-C₄H₉)₃); a sodium aluminum hydride compound such as sodium aluminum hydride (NaAlH₄), and sodium aluminum bis(2-methoxyethoxy)hydride ([(CH₃OCH₂CH₂O)₂AlH₂]Na).

Examples of the borane compound may include diborane; a borane complex such as borane-tetrahydrofurane complex, and borane-dimethyl sulfide (dimethyl sulfide-borane) complex; and 9-borabicyclo[3.3.1]nonane (9-BBN).

Examples of the aluminum hydride compound may include aluminum hydride (AlH₃) and diisobutylaluminum hydride ((i-C₄H₉)₂AlH).

Examples of the organosilicon compound may include a trialkylsilane such as triethylsilane, a diarylsilane such as diphenylsilane, and an aryldialkylsilane such as phenyldimethylsilane.

Examples of the organotin compound may include a trialkylstannan such as tri-n-butylstannan, a dialkylstannan such as di-n-butylstannan, and a diarylstannan such as diphenylstannan.

These reducing agents may be used alone or in combination. Among these reducing agents, a metal borohydride compound such as an alkali metal borohydride compound is preferred, and more preferably a sodium borohydride compound such as sodium borohydride (NaBH₄).

The amount of the reducing agents is, for example, 2 to 10 mol, preferably 3 to 5 mol, and more preferably 3.5 to 4.5 mol relative to 1 mol of the compound represented by the formula (5a).

The reducing agent may be used together with other reagents (or activators) depending on the nature (or species) or the compound represented by the formula (5A) and other factors. For example, in a case where the sodium borohydride compound such as sodium borohydride (NaBH₄) is used as a reducing agent, the reducing agent may be used together with a boron trifluoride-ether complex such as boron trifluoride-diethyl ether complex. The ratio of the activators is, for example, 0.1 to 10 mol, preferably 0.5 to 5 mol, and more preferably 0.8 to 1.2 mol relative to 1 mol of the reducing agent.

The reaction may be carried out in the absence or presence of inactive or inert solvent(s). The solvent may include, for example, water; an alcohol; an ether such as a cyclic ether and a chain ether; a hydrocarbon such as an aliphatic hydrocarbon, an alicyclic hydrocarbon, and an aromatic hydrocarbon.

Examples of the alcohol may include a C₁-6alcohol (or C₁₋₆alkanol) such as methanol, ethanol and isopropanol.

Examples of the cyclic ether may include dioxane and tetrahydrofuran (THF). Examples of the chain ether may include a dialkyl ether such as diethyl ether and diisopropyl ether; and a glycol ether. The glycol ether may include, for example, a (poly)alkylene glycol monoalkyl ether such as methyl cellosolve and methyl carbitol; and a (poly)alkylene glycol dialkyl ether such as dimethoxyethane.

The aliphatic hydrocarbon may include, for example, hexane and dodecane. The alicyclic hydrocarbon may include, for example, cyclohexane. The aromatic hydrocarbon may include, for example, benzene, toluene, and xylene.

These solvents may be used alone or in combination. A preferred solvent may be the ether, and a more preferred one may be the cyclic ether such as THF.

The reaction may be carried out in an atmosphere of an inert gas, for example, a nitrogen gas; and a rare gas such as helium and argon. The reaction temperature is, for example, 0 to 50° C., and preferably 5 to 35° C. The reaction time is not particularly limited to a specific time, and may be, for example, about 1 to 48 hours.

After the completion of the reaction, if necessary, the reaction mixture may be separated and purified by a conventional separation and purification means, for example, a method such as washing, extraction, filtration, dehydration, drying, concentration, decantation, recrystallization, reprecipitation, chromatography, and a combination of these methods.

(Compound Represented by Formula (2))

In a case where p1 and p2 each denote 1 or more, the compound represented by the formula (2) can be prepared by subjecting the compound represented by the formula (4) to an addition reaction with an alkylene oxide (alkylene carbonate or haloalkanol) corresponding to a straight- or branched-chain alkylene group represented by A^{2a} and A²b. The addition reaction of the alkylene oxide (alkylene carbonate or haloalkanol) may be carried out by a conventional manner, for example, a method according to the method described in WO 2013/022065, specifically, a method of reacting alkylene oxides corresponding to A^{2a} and A^{2b} such as ethylene oxide in the presence of a base catalyst such as potassium hydroxide.

In a case where p1 and p2 each denote 0, that is, the above addition reaction is not performed, the compound represented by the formula (4) may be used as the compound represented by the formula (2) to prepare a di(meth)acrylate compound represented by the formula (1).

(Preparation of Di(Meth)Acrylate Compound Represented by Formula (1))

The di(meth)acrylate compound represented by the formula (1) can be prepared by reacting a compound represented by the formula (2) (corresponding to the compound represented by formula (4), when p1 and p2 each denote 0) with compounds represented by the formulae (3a) and (3b) ((meth)acrylic acids or ester-forming derivatives thereof). In this description and claims, the term “ester-forming derivative” means an alkyl ester (or a lower alkyl ester), specifically, a C₁₋₄alkyl ester such as methyl ester and ethyl ester; an acid halide such as an acid chloride; and an acid anhydride unless otherwise noted.

The compound represented by the formula (2) may include, for example, a compound corresponding to a compound specifically exemplified as a di(meth)acrylate compound represented by the formula (1).

In the formulae (3a) and (3b), the halogen atom represented by X^{1a} and X^{1b} may include, for example, a chlorine atom, a bromine atom, and an iodine atom. The halogen atom may be preferably a chlorine atom and a bromine atom, and more preferably a chlorine atom. As examples of the alkoxy group represented by X^{1a} and X¹b, there may be mentioned a lower alkoxy group, for example, a straight- or branched-chain C₁₋₄alkoxy group such as methoxy group, ethoxy group, propoxy group, isopropoxy group, n-butoxy group, isobutoxy group, s-butoxy group, and t-butoxy group. The alkoxy group is preferably a C₁₋₂alkoxy group such as methoxy group. Preferred X¹a and X^{1b} each represent a hydroxy group.

The compounds represented by the formulae (3a) and (3b) may include, for example, (meth)acrylic acid or an anhydride thereof; a (meth)acrylic acid halide such as (meth)acrylic acid chloride and (meth)acrylic acid bromide; a (meth)acrylic acid alkyl ester, specifically a (meth)acrylic acid C₁₋₄alkyl ester such as methyl (meth)acrylate, ethyl (meth)acrylate, and t-butyl (meth)acrylate. These compounds represented by the formulae (3a) and (3b) may be a commercially available product. Among these compounds represented by the formulae (3a) and (3b),(meth)acrylic acid is preferred. The compounds represented by the formulae (3a) and (3b) may be different compounds from each other, and these are preferably the same compounds.

The ratio of total amount of the compounds represented by the formulae (3a) and (3b) is, for example, 1 to 10 mol, preferably 1.05 to 5 mol, more preferably 1.1 to 2 mol, and further preferably 1.2 to 1.5 mol relative to 1 mol of the hydroxy group of the compound represented by the formula (2).

In a case where each of X^{1a} and X^{1b} in the formulae (3a) and (3b) represents a halogen atom [in a case where each compound represented by the formulae (3a) and (3b) is a (meth)acrylic acid halide], the reaction may be carried out in the presence of a base to trap or acquire the hydrogen halide produced in the reaction. The base can be roughly classified into, for example, an inorganic base and an organic base.

As examples of the inorganic base, there may be mentioned a metal hydroxide, specifically an alkali metal or alkaline earth metal hydroxide such as sodium hydroxide and calcium hydroxide; a metal carbonate, specifically an alkali metal or alkaline earth metal carbonate such as sodium carbonate and calcium carbonate; a metal hydrogen carbonate (a metal bicarbonate), specifically an alkali metal or alkaline earth metal hydrogen carbonate such as sodium hydrogen carbonate.

Examples of the organic base may include an amine, specifically a trialkylamine such as triethylamine; an aromatic tertiary amine such as benzyldimethylamine; a heterocyclic amine such as pyridine and N-methylmorpholine.

These bases may be used alone or in combination. As the base, the amine, for example, the trialkylamine such as triethylamine is preferred. The amount of the base is not particularly limited to a specific one, and may be, for example, 1 to 2 mol, preferably 1.05 to 1.5 mol, and more preferably 1.1 to 1.2 mol relative to 1 mol of the (meth)acrylic acid halide.

In a case where each of X^{1a} and X^{1b} in the formulae (3a) and (3b) represents a hydroxy group or an alkoxy group [in a case where each compound represented by the formulae (3a) and (3b) is (meth)acrylic acid (or anhydride thereof), or (meth)acrylic acid alkyl ester], the reaction may be carried out in the presence of a conventional esterification catalyst. The catalyst may include an acid catalyst; a base catalyst; and a metal catalyst such as a metal alkoxide, specifically, a titanium(IV) alkoxide such as titanium(IV) tetraisopropoxide. Among these catalysts, the acid catalyst can be preferably used.

The acid catalyst is not particularly limited to a specific one, and may include an inorganic acid; an organic acid; a Lewis acid such as a boron trifluoride etherate, a tin tetrachloride; and a solid acid catalyst such as a cation exchange resin. These acid catalysts may be used alone or in combination of two or more. Further, these acid catalysts may be in the form of a hydrate.

As examples of the inorganic acid, there may be mentioned a strong acid, specifically a strong acid such as sulfuric acid, hydrochloric acid, nitric acid and phosphoric acid; a homo or heteropolyacid, specifically a homo or heteropolyacid such as tungstophosphoric acid, molybdophosphoric acid, tonguestosilic acid, and molybdosilicic acid.

As examples of the organic acid, there may be mentioned a sulfonic acid, specifically an alkanesulfonic acid such as methanesulfonic acid and ethanesulfonic acid; a fluoroalkanesulfonic acid such as trifluoromethanesulfonic acid; an arenesulfonic acid such as p-toluenesulfonic acid. As the acid catalyst, the arenesulfonic acid such as p-toluenesulfonic acid monohydrate is preferred.

The ratio of the catalyst is not particularly limited to a specific ratio, and, for example is 0.001 to 1 mol, and preferably 0.01 to 0.5 mol relative to 1 mol of the compound represented by the formula (2).

The reaction may be carried out in the presence of a polymerization inhibitor. The polymerization inhibitor may be added after the reaction is completed. The polymerization inhibitor may include, for example, benzoquinone; a hydroquinone compound such as hydroquinone, hydroquinone monomethyl ether (MEHQ), t-butylhydroquinone, and p-benzoquinone; a catechol such as p-t-butylcatechol and 2-methoxyphenol; an amine such as N,N-diethylhydroxylamine; 1,1-diphenyl-2-picrylhydrazyl; tri-p-nitrophenylmethyl; and phenothiazine. These polymerization inhibitors may be used alone or in combination of two or more. Among these polymerization inhibitors, the catechol such as 2-methoxyphenol is preferred.

The ratio of the polymerization inhibitor may be, for example, about 0.001 to 10 parts by mass relative to 100 parts by mass of the total amount of the compounds represented by the formulae (3a) and (3b). Further, the ratio of the polymerization inhibitor may be, for example, about 0.0001 to 0.1 parts by mass relative to 100 parts by mass of the di(meth)acrylate compound, which is a reaction product, represented by the formula (1).

The reaction may be carried out in the presence of solvent(s). The solvent may include, for example, a hydrocarbon, specifically an aliphatic hydrocarbon such as hexane and heptane, an alicyclic hydrocarbon such as cyclohexane, and an aromatic hydrocarbon such as toluene and xylene; a halogenated hydrocarbon, specifically a halogenated hydrocarbon such as methylene chloride, chloroform, 1,2-dichloroethane, and chlorobenzene; an ether, specifically a dialkyl ether such as diethyl ether, and a cyclic ether such as tetrahydrofuran (THF) and 1,4-dioxane; a ketone, specifically a ketone such as acetone and methylethylketone; a sulfoxide, specifically a sulfoxide such as dimethyl sulfoxide; an amide, specifically an amide such as N,N-dimethylformamide, N,N-dimethylacetamide, and N-methyl-2-pyrrolidone; and a nitrile such as acetonitrile. The solvent may be used alone or in combination. Among these solvents, an aromatic hydrocarbon such as toluene is preferred. The ratio of the solvent is not particularly limited to a specific ratio, and may for example be about 10 to 1000 parts by mass, and the ratio is preferably 50 to 150 parts by mass relative to 100 parts by mass of the total amount of the compounds represented by the formulae (2), (3a) and (3b).

The reaction temperature and reaction time may suitably be selected depending on the species of the raw materials and other factors. In a case where each of the compounds represented by the formulae (3a) and (3b) is a (meth)acrylic acid halide, the reaction temperature is, for example, −10° C. to 30° C., preferably 0 to 20° C., and more preferably 2 to 10° C. In a case where each of the compounds represented by the formulae (3a) and (3b) is a (meth)acrylic acid (or anhydride thereof), or (meth)acrylic acid alkyl ester, the reaction temperature is, for example, 50 to 150° C., preferably 80 to 130° C., and more preferably 100 to 120° C. The reaction may be carried out at the reflux temperature. The reaction time is not particularly limited to a specific time, and may be, for example, about 1 to 24 hours.

The reaction can be carried out in air or in an atmosphere of an inert gas such as a nitrogen gas; or a rare gas while stirring. The reaction may also be carried out under a normal or ordinary pressure, under a pressure, or under a reduced pressure. Further, in order to effectively prevent unexpected polymerization during the reaction, air may be blown into the liquid reaction mixture.

After the completion of the reaction, the produced di(meth)acrylate compound represented by the formula (1) may be separated and purified by a conventional means, for example, a separation and purification method such as neutralization, washing, dehydration, filtration, adsorption, concentration, extraction, crystallization, recrystallization, reprecipitation, centrifugal separation, and column chromatography, and a combination of these methods.

[Curable Composition and Cured Product Thereof]

The present disclosure includes a curable composition which contains the di(meth)acrylate compound represented by the formula (1) (the compound may be referred to as a first polyfunctional (meth)acrylate), and a cured product thereof. The curable composition contains at least the first polyfunctional (meth)acrylate, and may or may not contain other polymerization components. The other polymerization component may include, for example, a second polyfunctional (meth)acrylate different from the di(meth)acrylate compound represented by the formula (1); and a monofunctional polymerization component such as a monofunctional (meth)acrylate (or a reactive diluent).

(Second Polyfunctional (Meth)Acrylate)

The second polyfunctional (meth)acrylate is not particularly limited to a specific one, and may be a compound containing a plurality of (two or more) (meth)acryloyl groups. The number of (meth)acryloyl groups per molecule is, for example, 2 to 10, preferably 2 to 6, more preferably 2 to 4, further preferably 2 to 3, and particularly 2.

Examples of the second polyfunctional (meth)acrylate may include an epoxy (meth)acrylate (vinyl ester resin) such as an aliphatic epoxy (meth)acrylate, an alicyclic epoxy (meth)acrylate, an aromatic epoxy (meth)acrylate, and a poly(meth)acrylate of Novolak (Novolac) epoxy resin; an urethane (meth)acrylate; a polyester (meth)acrylate (a poly(meth)acrylate of polyester polyol with 2 or more hydroxyl groups); an alkylene glycol di(meth)acrylate; a polyalkylene glycol di(meth)acrylate; a di(meth)acrylate of an alicyclic diol; a di(meth)acrylate of a biphenol or a bisphenol, or an alkylene oxide (an alkylene carbonate or haloalkanol) adduct thereof; a poly(meth)acrylate of a low molecular weight polyol compound with about 3 to 6 hydroxyl groups or an alkylene oxide (alkylene carbonate or haloalkanol) adduct thereof. These second polyfunctional (meth)acrylate may be used alone or in combination of two or more. These second polyfunctional (meth)acrylate may be a commercially available product.

The aliphatic epoxy (meth)acrylate may include, for example, a di(meth)acrylate of (poly)alkylene glycol diglycidyl ether such as di(meth)acrylate of 1,6-hexanediol diglycidyl ether, and di(meth)acrylate of polypropylene glycol diglycidyl ether.

The alicyclic epoxy (meth)acrylate may include, for example, a di(meth)acrylate of an epoxy compound with a C₅₋₁₀aliphatic ring such as di(meth)acrylate of 1,4-cyclohexanedimethanol diglycidyl ether.

The aromatic epoxy (meth)acrylate may include, for example, a di(meth)acrylate of diglycidyl ether of a bisphenol, or a biphenol or alkylene oxide (alkylene carbonate or haloalkanol) adduct thereof, such as di(meth)acrylate of bisphenol A diglycidyl ether. Examples of the bisphenol may include bisphenol A, bisphenol F, bisphenol AD, and bisphenol S. Examples of the biphenol may include p,p′-biphenol, m,m′-biphenyl, and o,o′-biphenol.

The alkylene glycol di(meth)acrylate may include, for example, a C₂₋₁₀alkylene glycol di(meth)acrylate such as ethylene glycol di(meth)acrylate, and butanediol di(meth)acrylate.

The polyalkylene glycol di(meth)acrylate may include, for example, a di- to hexa-C₂₋₁₀alkylene glycol di(meth)acrylate such as diethylene glycol di(meth)acrylate.

The di(meth)acrylate of an alicyclic diol may include, for example, a di(meth)acrylate of a C₅₋₁₀aliphatic ring-containing diol compound such as di(meth)acrylate of 1,4-cyclohexanedimethanol.

In the di(meth)acrylate of a biphenol or a bisphenol, or an alkylene oxide (alkylene carbonate or haloalkanol) adduct thereof, the biphenol or bisphenol may include, for example, the biphenol or the bisphenol exemplified in the section of the aromatic epoxy (meth)acrylate; and a 9,9-bis[hydroxyaryl]fluorene.

The poly(meth)acrylate of a low molecular weight polyol compound with about 3 to 6 hydroxyl groups or an alkylene oxide (alkylene carbonate or haloalkanol) adduct thereof may include, for example, glycerin tri(meth)acrylate, diglycerin tetra(meth)acrylate, trimethylolethane tri(meth)acrylate, trimethylolpropane tri(meth)acrylate, pentaerythritol tri(meth)acrylate, pentaerythritol tetra(meth)acrylate, dipentaerythritol penta(meth)acrylate, dipentaerythritol hexa(meth)acrylate, and a sorbitol tri- to hexa-(meth)acrylate.

Among these second polyfunctional (meth)acrylates, the di(meth)acrylate of a biphenol or a bisphenol, or an alkylene oxide (alkylene carbonate or haloalkanol) adduct thereof is preferred, and the di(meth)acrylate of 9,9-bis [hydroxyaryl]fluorene or an alkylene oxide (an alkylene carbonate or haloalkanol) adduct thereof represented by the following formula (7) is more preferred from the viewpoint of the excellent balance of the properties such as high refractive index, high heat resistance, flexibility (toughness) and high curability.

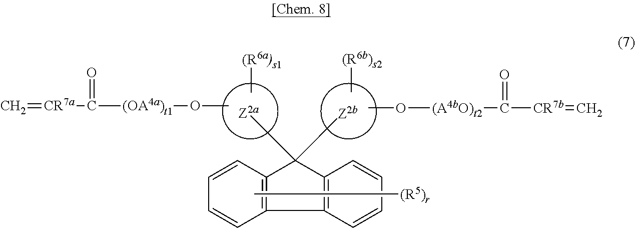

In the formula (7), Z^{2a} and Z^{2b} independently represent an arene ring,

R⁵ represents a substituent, r denotes an integer of 0 to 8,

R^{6a} and R^{6b} independently represent a substituent, s1 and s2 independently denote an integer of not less than 0,

A^{4a} and A^{4b} independently represent a straight- or branched-chain alkylene group, t1 and t2 independently denote an integer of not less than 0, and

R^{7a} and R^{7b} independently represent a hydrogen atom or a methyl group.

In the formula (7), the arene ring represented by Z^{2a} and Z^{2b} may include, for example, an arene ring exemplified as Z^{1a} and Z^{1b} in the formula (1). Each of the rings Z^{2a} and Z^{2b} is preferably a C₆₋₁₂arene ring such as a benzene ring, a naphthalene ring, and a biphenyl ring, more preferably a C₆₋₁₀arene ring such as a benzene ring and a naphthalene ring, and particularly a benzene ring.

The substituent (non-reactive substituent or non-polymerizable substituent) represented by R⁵ may include, for example, a hydrocarbon group such as an alkyl group and an aryl group; a cyano group; and a halogen atom such as a fluorine atom, a chlorine atom, a bromine atom, and an iodine atom. The alkyl group may include, for example, a straight- or branched-chain C₁₋₆alkyl group such as methyl group, ethyl group, propyl group, isopropyl group, n-butyl group, and t-butyl group. The aryl group may include, for example, a C₆₋₁₀aryl group such as phenyl group.

In a case of denoting r of not less than 1, a preferred group R⁵ is an alkyl group, a cyano group and a halogen atom, more preferably an alkyl group, and particularly a straight- or branched-chain C₁₋₄alkyl group such as methyl group.

The number r of the substituents R⁵ may for example be an integer of about 0 to 7; and a preferred range of the number r is an integer of 0 to 6, an integer of 0 to 4, an integer of 0 to 3, and an integer of 0 to 2 in a stepwise manner; the number r is more preferably 0 or 1, and particularly 0. When r denotes 2 or more, the species of the two or more group R⁵ may be the same or different from each other. The substitution positions of the group R⁵ are not particularly limited to a specific one and the group R⁵ may be, for example, bonded at any one of 2- to 7-positions of the fluorene ring, preferably at 2-, 3- and/or 7-positions of the fluorene ring, and more preferably at 2-position or 2,7-positions of the fluorene ring.

The substituents represented by R^{6a} and R^{6b} (non-reactive substituent or non-polymerizable substituent) may include, for example, the same group as the substituents exemplified as R^{1a} and R^{1b} in the formula (1).

Representative examples of these groups R^{6a} and R^{6b} may include, for example, the hydrocarbon group; the alkoxy group; the acyl group; the nitro group; the cyano group; and the substituted amino group. In a case where s1 denotes not less than 1, the group R^{6a} preferably is the alkyl group, the aryl group and the alkoxy group, specifically a straight- or branched-chain C₁₋₆alkyl group and a straight- or branched-chain C₁₋₄alkoxy group such as methoxy group.

Among these substituents, the alkyl group and the aryl group are preferred, particularly a straight- or branched-chain C₁₋₄alkyl group such as methyl group; and a C₆₋₁₀aryl group such as phenyl group are preferred. The same relationship between s1 and R^{6a} also applies to s2 and R^{6b}. When the group R^{6a} is the aryl group, the group R^{6a} may form the ring-assemblies arene ring together with the ring Z²a. The same relationship between R^{6a} and Z^{2a} also applies to R^{6b} and Z²b.

Each of numbers s1 and s2 of the substituents of the groups R^{6a} and R^{6b} may denote an integer of not less than 0, and may suitably be selected according to the species of the ring Z^{2a} or Z²b. The numbers s1 and s2 each denote, for example, an integer of about 0 to 8; and preferably an integer of 0 to 4, an integer of 0 to 3, and an integer of 0 to 2 in a stepwise manner; the numbers s1 and s2 are more preferably 0 or 1, and particularly 0.

The numbers s1 and s2 may be different from each other, and the same numbers are preferred. In a case where the number s1 of substituents denotes 2 or more, the species of the two or more groups R^{6a} may be the same or different from each other. The same relationship between s1 and R^{6a} also applies to s2 and R^{6b}. Further, the species of the groups R^{6a} and R^{6b} are the same or different from each other. The substitution positions of the groups R^{6a} and R^{6b} are not particularly limited to a specific one. The groups R^{6a} and R^{6b} may be bonded to positions other than the bonding positions of the rings Z^{2a} and Z²b with the ether bond (—O—) and the 9-position of the fluorene ring. In the rings Z^{2a} and Z²b, the groups R^{6a} and R^{6b} are preferably bonded in relation to the ortho position (a carbon atom adjacent to the bonding position of the ether bond) with respect to the ether bond (—O—).

The straight- or branched-chain alkylene group represented by A⁴a and A⁴b, including preferable embodiments, is, for example, the same as the alkylene group exemplified as A^{2a} and A^{2b} in the formula (1), and ethylene group is particularly preferred. The species of the groups A⁴a and A^{4b} may be different from each other, and the same species are preferred.

The repeating numbers t1 and t2 of oxyalkylene groups (OA⁴a) and (OA⁴b) each may for example be selected from a range of about 0 to 20. In applications where high refractive index and heat resistance are required, a preferred range of the repeating numbers t1 and t2 each are 0 to 15, 0 to 10, 0 to 6, and 0 to 2 in a stepwise manner; and particularly 0 to 1. In applications where low viscosity or flexibility (toughness) is required, a preferred range of the repeating numbers t1 and t2 each are 1 to 10, 3 to 8, and 4 to 7 in a stepwise manner; and particularly 5 to 6. In a case where t1 denotes 2 or more, the species of the two or more groups A⁴a in a polyoxyalkylene group [-(OA⁴a)ₜ₁-] may be different from each other, and the same species are preferred. The same relationship between t1 and A⁴a also applies to t2 and A⁴b.

The numbers t1 and t2 may be the same or different from each other. The repeating numbers t1 and t2 each may be an average (or arithmetic average, arithmetical average), that is, an average addition molar amount; and the range of the average, including preferred embodiments (ranges), is within the above-mentioned integer range.

The total number of the repeating numbers t1 and t2 means a total number (or an average of total addition molar amount) of oxyalkylene groups (OA⁴a) and (OA⁴b) per molecule of the di(meth)acrylate compound represented by the formula (7), and may be simply referred to as t1+t2. The total number t1+t2 may for example be selected from a range of about o to 30. In applications where high refractive index and heat resistance are required, a preferred range of t1+t2 is 0 to 20, 0 to 12, and 0 to 4 in a stepwise manner; and particularly 0 to 2. In applications where low viscosity or flexibility (toughness) is required, a preferred range of t1+t2 is 2 to 20, 6 to 16, and 8 to 14 in a stepwise manner, and particularly 10 to 12. The total number t1+t2 may be an integer as mentioned above, and may be the average of total addition molar amount. The range of the average of total addition molar amount, including preferred embodiments (ranges), is within the above-mentioned integer range. The total number t1+t2 can be measured according to the measuring method of the total number p1+p2 in the formula (1).

An excessively large repeating number t1, t2, or t1+t2 may make it difficult to improve the refractive index or heat resistance, and an excessively small repeating number t1, t2, or t1+t2 may make it difficult to improve the handleability or flexibility.

The substitution positions of the groups [—O-(A^{4a}O)ₜ₁-] and [—O-(A^{4b}O) t2-] on the rings Z^{2a} and Z^{2b} are not particularly limited to a specific position. In a case where the ring Z^{2a} is a benzene ring, the substitution position of the group [—O-(A^{4a}O) t1-] may be any one of 2-position, 3-position, or 4-position of the phenyl group bonded to 9-position of the fluorene ring; preferably 3-position or 4-position, and particularly 4-position. In a case where the ring Z^{2a} is a naphthalene ring, the substitution position of the group [—O-(A^{4a}O)ₜ₁-] is preferably any one of 5- to 8-positions of the naphthyl group bonded to 9-position of the fluorene ring. In particular, it is preferable that 1-position or 2-position of the naphthalene ring is bonded to 9-position of the fluorene ring (i.e., the fluorene ring has 1-naphthyl or 2-naphthyl substituent); and the group [—O-(A^{1a}O) n1-] and 9-position of the fluorene ring are preferably bonded to the naphthalene ring at a positional relationship of 1,5-position, 2,6-position, and particularly 2,6-position. When the ring Z^{2a} is a ring-assemblies arene ring, the substitution position of the group [—O-(A^{4a}O)ₜ₁-] relative to the ring-assemblies arene ring is not limited to a specific position. For example, When the ring Z^{2a} is a biphenyl ring (or the ring Z^{2a} is a benzene ring, s1 denotes 1, and R^{6a} is a phenyl group), 3-position of the biphenyl ring may preferably be bonded to 9-position of the fluorene, and further the substitution position of the group [—O-(A^{4a}O)ₜ₁-] may for example be either 6-position or 4′-position, and particularly 6-position of the biphenyl group.

The groups R^{7a} and R^{7b} each may represent either a hydrogen atom or a methyl group, and preferably a hydrogen atom from a viewpoint of easily improving or increasing reactivity (or curability) and refractive index. The species of the groups R^{7a} and R^{7b} may be the same or different from each other, and the same species are preferred.

Representative examples of the di(meth)acrylate compound represented by the formula (7) may include a di(meth)acrylate compound of a 9,9-bis[hydroxyaryl]fluorene [or alkylene oxide (alkylene carbonate or haloalkanol) adduct thereof] in which each of Z^{2a} and Z^{2b} represents a C₆₋₁₂arene ring, each of R^{6a} and R^{6b} represents a hydrocarbon group, each of s1 and s2 denotes an integer of 0 to 2, each of A⁴a and A^{4b} represents a straight- or branched-chain C₂-4alkylene group, and each of t1 and t2 denotes an integer of 0 to 10.

Representative examples of the 9,9-bis[hydroxyaryl]fluorene forming the di(meth)acrylate compound represented by the formula (7) may include a 9,9-bis(hydroxyphenyl)fluorene, a 9,9-bis(alkyl-hydroxyphenyl)fluorene, a 9,9-bis(aryl-hydroxyphenyl)fluorene, and a 9,9-bis(hydroxynaphthyl)fluorene.

The 9,9-bis(hydroxyphenyl)fluorene may include, for example, 9,9-bis(4-hydroxyphenyl)fluorene.

The 9,9-bis(alkyl-hydroxyphenyl)fluorene may include, for example, a 9,9-bis[(mono- or di-)C₁₋₄alkyl-hydroxyphenyl]fluorene such as 9,9-bis(4-hydroxy-3-methylphenyl)fluorene, and 9,9-bis(4-hydroxy-3,5-dimethylphenyl)fluorene.

The 9,9-bis(aryl-hydroxyphenyl)fluorene may include, for example, a 9,9-bis(C₆₋₁₀aryl-hydroxyphenyl)fluorene such as 9,9-bis(4-hydroxy-3-phenylphenyl)fluorene.

The 9,9-bis(hydroxynaphthyl)fluorene may include, for example, 9,9-bis(6-hydroxy-2-naphthyl)fluorene, and 9,9-bis(5-hydroxy-1-naphthyl)fluorene.

Representative examples of the alkylene oxide (alkylene carbonate or haloalkanol), that may be added to the 9,9-bis[hydroxyaryl]fluorene, may include a C₂₋₃alkylene oxide (C₂₋₃alkylene carbonate or C₂₋₃haloalkanol) such as ethylene oxide and propylene oxide. The addition molar amount (or an average of the addition molar amount) of the alkylene oxide (alkylene carbonate or haloalkanol) t1+t2 may for example be selected from a range of about 0 to 20. In applications where high refractive index and heat resistance are required, a preferred range of t1+t2 is 0 to 2; and in applications where low viscosity or flexibility (toughness) is required, a preferred range of t1+t2 is 9 to 13.

These di(meth)acrylate compounds represented by the formula (7), may be used alone or in combination of two or more. Among these compounds, the preferred compound is a compound in which Z^{2a} and Z^{2b} each represent a benzene ring, A⁴a and A^{4b} each represent a straight- or branched-chain C₂₋₃alkylene group, and t1+t2 denotes 10 to 12.

In the curable composition containing the second polyfunctional (meth)acrylate, the ratio of the di(meth)acrylate compound represented by the formula (7) relative to the total amount of the second polyfunctional (meth)acrylate may for example be selected from a range of about 30 to 100; by mass; a preferred range of the ratio is not less than 50% by mass, not less than 70% by mass, and not less than 90% by mass in a stepwise manner; and more preferably substantially 100% by mass, specifically, the second polyfunctional (meth)acrylate only contains the di(meth)acrylate compound represented by the formula (7).

In the curable composition containing the compound represented by the formula (7), the mass ratio of the compound represented by the formula (1) relative to the compound represented by the formula (7) may for example be selected from a range of about 10/90 to 90/10, e.g., about 20/80 to 80/20; and the mass ratio is preferably 30/70 to 70/30 in terms of the former/the latter. The mass ratio may be selected according to the application from the viewpoint of adjusting the balance of the properties such as refractive index, heat resistance, flexibility (or toughness) and curability. In applications where higher refractive index is required, a preferred range of the mass ratio is 50/50 to 80/20, and more preferably 60/40 to 75/25; and in applications where more flexibility (toughness) and/or handleability (lower viscosity or higher solubility) is required, a preferred range of the mass ratio is 20/80 to 50/50, and more preferably 25/75 to 40/60. When the ratio of the compound represented by the formula (7) is excessively high, the resulting curable composition may not have a sufficiently high refractive index, and when the ratio of the compound represented by the formula (7) is excessively low, it may be difficult to improve the flexibility (toughness) and/or handleability (viscosity or solubility) of the resulting curable composition.

The ratio of the first polyfunctional (meth)acrylate represented by the formula (1) relative to the total amount of the first and second polyfunctional (meth)acrylates may for example be not less than 10% by mass, specifically, may for example be selected from a range of about 30 to 100% by mass. A preferred range of the ratio is not less than 50% by mass, not less than 60; by mass, not less than 70; by mass, and not less than 80% by mass in a stepwise manner; and more preferably not less than 90% by mass. In particular, it is further preferred that the ratio be substantially 100% by mass, specifically, the polyfunctional polymerization component only contains the first polyfunctional (meth)acrylate. The ratio may for example be selected from a range of about 60 to 99% by mass, and specifically may be about 80 to 97% by mass. In a case where the ratio of the first polyfunctional (meth)acrylate is excessively low, the refractive index and heat resistance of the resulting curable composition may decrease.

(Monofunctional Polymerization Component)

The monofunctional polymerization component (or reactive diluent) may be a compound having one polymerizable group (or polymerizable unsaturated bond) such as an alkenyl group, e.g., vinyl group and an allyl group; and (meth)acryloyl group. Specifically, the monofunctional polymerization component may include a monofunctional vinyl monomer; and a monofunctional (meth)acrylic monomer. The monofunctional vinyl monomer may include, for example, a α-olefinic monomer such as ethylene and propylene; a styrenic monomer such as styrene, α-methylstyrene, and vinyltoluene; a vinylester monomer such as vinyl acetate; and N-vinyl pyrrolidone. The monofunctional (meth)acrylic monomer may include, for example, (meth)acrylic acid; (meth)acrylamide; a N-substituted (meth)acryl amide such as N-methylol (meth)acrylamide, and N,N-dimethyl (meth)acrylamide; (meth)acrylonitrile; and a monofunctional (meth)acrylate.

These monofunctional polymerization components may be used alone or in combination of two or more. Among these monofunctional polymerization components, the monofunctional (meth)acrylic-series monomer, and particularly the monofunctional (meth)acrylate is preferred.

Examples of the monofunctional (meth)acrylate may include an aliphatic monofunctional (meth)acrylate; an alicyclic monofunctional (meth)acrylate; an aromatic monofunctional (meth)acrylate; and monofunctional (meth)acrylate with a sulfur atom. These monofunctional (meth)acrylates may be used alone or in combination of two or more.

Examples of the aliphatic monofunctional (meth)acrylate may include a C₁₋₂₀alkyl (meth)acrylate such as methyl (meth)acrylate, n-butyl (meth)acrylate, and 2-ethylhexyl (meth)acrylate.

Examples of the alicyclic monofunctional (meth)acrylate may include a C₅₋₁₀cycloalkyl (meth)acrylate such as cyclohexyl (meth)acrylate; a bridged (crosslinked) ring (meth)acrylate such as dicyclopentenyl (meth)acrylate, and isobornyl (meth)acrylate.

Examples of the aromatic monofunctional (meth)acrylate may include an aryl (meth)acrylate such as phenyl (meth)acrylate; an aralkyl (meth)acrylate such as benzyl (meth)acrylate; an aryloxyalkyl (meth)acrylate, specifically a C₆₋₁₂aryloxyC₂₋₄alkyl (meth)acrylate such as 2-phenoxyethyl (meth)acrylate, 2-(2-naphthoxy)ethyl (meth)acrylate, and 2-(o-phenylphenoxy)ethyl (meth)acrylate; a mono(meth)acrylate of a bisphenol or a biphenol (or alkylene oxide adduct thereof) such as mono(meth)acrylate of ethylene oxide adduct of bisphenol A; and a (meth)acrylate with a fluorene skeleton such as 9-(meth)acryloyloxymethyl fluorene.

Examples of the monofunctional (meth)acrylate with a sulfur atom may include an alkylthio(meth)acrylate; an arylthio(meth)acrylate; an aralkylthio(meth)acrylate; and an arylthioalkyl (meth)acrylate. Examples of the alkylthio(meth)acrylate may include a C₁₋₆alkylthio(meth)acrylate such as methylthio(meth)acrylate. Examples of the arylthio(meth)acrylate may include a C₆₋₁₀arylthio(meth)acrylate such as phenylthio(meth)acrylate. Examples of the aralkylthio(meth)acrylate may include a C₆₋₁₀arylC₁₋₆alkylthio(meth)acrylate such as benzylthio(meth)acrylate. Examples of the arylthioalkyl (meth)acrylate may include a C₆₋₁₀arylthioC₂₋₄alkyl (meth)acrylate such as phenylthioethyl (meth)acrylate.

Among these monofunctional (meth)acrylates, from the viewpoint of easily decreasing the viscosity while maintaining the high refractive index, the preferred monofunctional (meth)acrylate is an aromatic monofunctional (meth)acrylate, and more preferably a compound represented by the following formula (8).

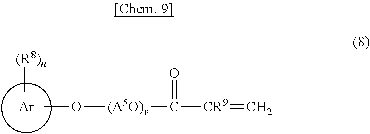

In the formula (8), Ar represents an arene ring,

R⁸ represents a substituent, u denotes an integer of not less than 0,

A⁵ represents a straight- or branched-chain alkylene group, v denotes an integer of not less than 0, and

R⁹ represents a hydrogen atom or a methyl group.

In the formula (8), examples of the arene ring represented by Ar may include the arene ring exemplified as Z^{1a} and Z^{1b} in the formula (1). The ring Ar is preferably a C₆₋₁₂arene ring such as a benzene ring, a naphthalene ring, and a biphenyl ring; more preferably a benzene ring and a biphenyl ring; and particularly a biphenyl ring from the viewpoint of having a high refractive index and an excellent curability in combination with (meth)acylate represented by the formula (1).

The substituent represented by R⁸ may include a hydrocarbon group, and a preferred example of the substituent is an alkyl group. The alkyl group may include, for example, a straight- or branched-chain C₁₋₁₂alkyl group such as methyl group, ethyl group, n-propyl group, isopropyl group, n-butyl group, isobutyl group, s-butyl group, t-butyl group, pentyl group, neopentyl group, hexyl group, octyl group, 2-ethylhexyl group, and decyl group. The preferred alkyl group may be a straight- or branched-chain C₁₋₉alkyl group, a straight- or branched-chain C₁₋₆alkyl group, and a straight- or branched-chain C₁₋₄alkyl group in a stepwise manner.

The number u of the substituents of the group R⁸ may denote 0 or an integer of not less than 1, and may suitably be selected according to the species of the ring Ar, and other factors. The number u may denote, for example, an integer of about 0 to 4, preferably an integer of about 0 to 2, more preferably 0 or 1, and particularly 0. In a case where u denotes 2 or more, the species of the two or more groups R⁸ may be the same or different from each other. Further, the substitution position of the group R⁸ is not particularly limited to the specific position.

The straight- or branched-chain alkylene group represented by A⁵ is, for example, the same as the alkylene group, including preferred embodiments, exemplified as A^{2a} and A^{2b} in the formula (1), and ethylene group is particularly preferred.

The repeating number v of the oxyalkylene group (OA⁵) may for example be selected from a range of about 0 to 10; the repeating number v is preferably 0 to 4, 1 to 3, and 1 to 2 in a stepwise manner; and particularly 1. An excessively large repeating number v may make it difficult to improve the refractive index and heat resistance, and an excessively small repeating number v may make it difficult to improve the handleability. When v denotes 2 or more, the species of the two or more groups A⁵ in a (poly)oxyalkylene group [-(OA⁵)ᵥ-] may be different from each other, and the same species are preferred.

The group R⁹ may represent either a hydrogen atom or a methyl group, and preferably a hydrogen atom from a viewpoint of improving or increasing reactivity (or curability) and refractive index.

The bonding position of the group [—O-(A⁵O)ᵥ—CO—CR⁹═CH₂] on the ring Ar is not particularly limited to a specific position, and when the ring Ar is a biphenyl ring, the bonding position of the group [—O-(A⁵O)ᵥ—CO—CR⁹═CH₂] is preferably 2-position of the biphenyl ring.

Representative examples of the compound represented by the formula (8) may include a compound in which Ar represents a C₆₋₁₂arene ring such as a benzene ring, a naphthalene ring, and a biphenyl ring; R⁸ represents a hydrocarbon group such as an alkyl group; u denotes an integer of 0 to 2; A⁵ represents a straight- or branched-chain C₂₋₄alkylene group; and v denotes an integer of 1 to 4. Specific examples of the compound represented by the formula (8) may include a compound in which Ar represents a benzene ring; A⁵ represents a straight- or branched-chain C₂₋₃alkylene group; and v denotes an integer of 1 to 2, specifically, a phenoxyC₂₋₃alkyl (meth)acrylate such as 2-phenoxyethyl (meth)acrylate; a compound in which Ar represents a biphenyl ring; A⁵ represents a straight- or branched-chain C₂₋₃alkylene group; and v denotes an integer of 1 to 2, specifically, a biphenylyloxyC₂₋₃alkyl (meth)acrylate such as 2-(o-phenylphenoxy)ethyl (meth)acrylate; and a compound in which Ar represents a naphthalene ring; A⁵ represents a straight- or branched-chain C₂₋₃alkylene group; and v denotes an integer of 1 to 2, specifically, a naphthoxy C₂₋₃alkyl (meth)acrylate such as 2-(2-naphthoxy)ethyl (meth)acrylate.

These compounds represented by the formula (8) may be used alone or in combination of two or more. Among these compounds, biphenylyloxyC₂₋₃alkyl (meth)acrylate is preferred from a viewpoint of easily being compatible the high refractive index with the curability.

In the curable composition containing the compound represented by the formula (8), the mass ratio of the compound represented by the formula (1) relative to the compound represented by the formula (8) may be selected from a range of about 10/90 to 95/5, e.g., about 30/70 to 90/10 in terms of the former/the latter, the mass ratio is preferably 50/50 to 85/15, 60/40 to 80/20, and 65/35 to 75/25 in terms of the former/the latter in a stepwise manner. When the ratio of the compound represented by the formula (8) is excessively high, the resulting curable composition may have a low refractive index and curability, and when the ratio of the compound represented by the formula (8) is excessively low, the handleability of the resulting curable composition may not sufficiently improved.

In the curable composition containing the compound represented by the formula (8), the ratio of the compound represented by the formula (8) relative to the total amount of the monofunctional (meth)acrylate may for example be selected from a range of about 30 to 100% by mass; a preferred range of the ratio is not less than 50% by mass, not less than 70% by mass, and not less than 90% by mass in a stepwise manner; and the ratio is more preferably substantially 100% by mass, specifically, the monofunctional (meth)acrylate only contains the compound represented by the formula (8). Further, in the curable composition containing the monofunctional (meth)acrylate, the ratio of the monofunctional (meth)acrylate relative to the total monofunctional polymerization component may for example be selected from a range of about 30 to 100% by mass; a preferred range of the ratio is not less than 50% by mass, not less than 70% by mass, and not less than 90% by mass in a stepwise manner; and more preferably substantially 100% by mass.

Regarding the ratio of monofunctional polymerization component, in the curable composition containing the monofunctional polymerization component, the mass ratio of the total polyfunctional (meth)acrylate (total amount of the first and second polyfunctional (meth)acrylates) relative to the monofunctional polymerization component may for example be about 10/90 to 95/5; and the mass ratio is preferably 30/70 to 90/10, 50/50 to 85/15, and 60/40 to 80/20 in terms of the former/the latter in a stepwise manner. The ratio may be the same as the ratio of the total amount of the polyfunctional (meth)acrylate relative to the monofunctional (meth)acrylate, or the ratio of the total amount of the polyfunctional (meth)acrylate relative to the compound represented by the formula (8). When the ratio of the monofunctional polymerization component, particularly the monofunctional (meth)acrylate such as the compound represented by the formula (8), is excessively high, the resulting curable composition may not have a sufficiently high refractive index, and when the ratio of the monofunctional polymerization component is excessively low, it may be difficult to improve the handleability (lower viscosity) of the resulting curable composition.

(Components Other than Polymerization Components)

The curable composition may further contain, in addition to the polymerization component (or monomer component), a polymerization initiator, a solvent, an additive and others.

The polymerization initiator may be a thermal polymerization initiator (thermal radical generator) or a photopolymerization initiator (photoradical generator).

The thermal polymerization initiator may include, for example, an organic peroxide and an azo compound. The organic peroxide may include, for example, a dialkyl peroxide such as di-t-butyl peroxide; a diacyl peroxide such as lauroyl peroxide and benzoyl peroxide; a peracid (a peroxy acid) (or a peracid ester) such as t-butyl hydroperoxide, cumene hydroperoxide and t-butyl peracetate; a ketone peroxide; a peroxycarbonate; and a peroxyketal. The azo compound may include, for example, an azonitrile compound such as 2,2′-azobis(isobutyronitrile); an azoamide compound; and an azoamidine compound. These thermal polymerization initiators may be used alone or in combination of two or more.

The photopolymerization initiator may include, for example, a benzoin, specifically a benzoin alkyl ether such as benzoin and benzoin ethyl ether; an acetophenone compound such as acetophenone and 2-hydroxy-2-methyl-1-phenylpropane-1-one; an aminoacetophenone such as 2-methyl-1-[4-(methylthio)phenyl]-2-morpholinoaminopropanone-1; an anthraquinone compound such as anthraquinone and 2-methylanthraquinone; a thioxanthone such as 2,4-dimethylthioxanthone, 2,4-diethylthioxanthone, and 2-chlorothioxanthone; a ketal such as acetophenone dimethyl ketal and benzyl dimethyl ketal; a benzophenone compound such as benzophenone; and a xanthone. These photopolymerization initiators may be used alone or in combination of two or more.

The ratio of the polymerization initiator (thermal and/or photopolymerization initiator) relative to 100 parts by mass of the total amount of the polymerization component may for example be 0.1 to 15 parts by mass, preferably 0.5 to 10 parts by mass, more preferably 1 to 8 parts by mass, and further preferably 2 to 5 parts by mass.

Furthermore, the photopolymerization initiator may be combined with a photosensitizer. Representative examples of the photosensitizer may include a conventional photosensitizer such as a tertiary amine. Examples of the tertiary amine may include a trialkylamine; a trialkanolamine such as triethanolamine; a dialkylaminobenzoic acid alkyl ester, specifically an ethyl N,N-dimethylaminobenzoate such as ethyl p-(dimethylamino)benzoate, and an amyl N,N-dimethylaminobenzoate such as amyl p-(dimethylamino)benzoate; a bis(dialkylamino)benzophenone such as 4,4-bis(diethylamino)benzophenone; and a dialkylaminobenzophenone such as 4-(dimethylamino)benzophenone. These photosensitizers may be used alone or in combination of two or more.

The ratio of the photosensitizer relative to 100 parts by mass of the polymerization initiator may for example be 1 to 200 parts by mass, preferably 5 to 150 parts by mass, and more preferably 10 to 100 parts by mass.

The curable composition does not require to include solvent(s). If necessary, the curable composition may include solvent(s), in order to adjust the handleability thereof, since the di(meth)acrylate compound represented by the formula (1) has unexpectedly high solubility. The solvent is not limited to a specific one, and may include, for example, a hydrocarbon, specifically an aliphatic hydrocarbon such as hexane and heptane, an alicyclic hydrocarbon such as cyclohexane, and an aromatic hydrocarbon such as toluene and xylene; a halogenated hydrocarbon, specifically a halogenated hydrocarbon such as methylene chloride, chloroform, 1,2-dichloroethane, and chlorobenzene; an ether, specifically a chain ether such as diethyl ether, and a cyclic ether such as tetrahydrofuran and 1,4-dioxane; a ketone, specifically a dialkylketone such as acetone, methylethylketone (MEK) and methylisobuthylketone (MIBK), and a cyclic ketone such as cyclohexanone; an ester, specifically an acetate (an acetic acid ester) such as methyl acetate, ethyl acetate, and butyl acetate; a glycol ether acetate, specifically (poly)alkylene glycol monoalkyl ether acetates such as propylene glycol monomethyl ether acetate (PGMEA), and diethylene glycol monobuthyl ether acetate; a sulfoxide, specifically a sulfoxide such as dimethyl sulfoxide; an amide, specifically an amide such as dimethylformamide (DMF), dimethylacetamide, and N-methyl-2-pyrrolidone; a nitrile, specifically a nitrile such as acetonitrile. These solvents can also be used alone or as a mixed solvent in combination of two or more. Among these solvents, a hydrocarbon, a ketone, an ester, a glycol ether acetate, and an amide are preferred, and an aromatic hydrocarbon, a ketone, an acetate (an acetic acid ester), and an amide are more preferred.

The ratio (amount) of the solvent is not particularly limited to a specific ratio, and may be adjusted so that a solid (nonvolatile) content (components other than the solvent) is, for example, about 0.1 to 50% by mass, concretely about 20 to 50% by mass, preferably 25 to 40% by mass, and more preferably 30 to 35% by mass relative to the total curable composition.

The curable composition may contain conventional additives. Examples of such an additive may include a colorant, a stabilizer, a filler, an antistatic agent, a flame retardant, a surfactant, a plasticizer, a curing agent, and a polymerization inhibitor. The stabilizer may include, for example, a heat stabilizer, an antioxidant, and an ultraviolet absorbing agent. These additives may be used alone or in combination of two or more.

The total ratio (amount) of the additive(s) relative to the total curable composition may for example be not more than 30% by mass; the total ratio is preferably not more than 20% by mass, not more than 10% by mass, and not more than 5% by mass in a stepwise manner. The total ratio (amount) of the additive(s) may be 0.001 to 15% by mass, and concretely 0.01 to 3% by mass.

(Cured Product)

The curable composition of the present disclosure is easily cured by applying active energy (or active energy rays) to produce a cured product. As the active energy, thermal energy and/or light energy such as ultraviolet rays and X-rays is(are) useful.

In a heat treatment using heat energy, the heating temperature is, for example, 50 to 200° C., preferably 60 to 150° C., and more preferably 70 to 120° C.

In a light irradiation using light energy such as ultraviolet rays, the intensity (amount) of light irradiation energy can be suitably selected depending on the applications, and is, for example, 50 to 10000 mJ/cm², preferably 70 to 8000 mJ/cm², more preferably 100 to 5000 mJ/cm², and particularly 500 to 3000 mJ/cm².

The shape of the cured product is not particularly limited to a specific one, and the cured product may have a three-dimensional structure such as a lens shape and a tubular shape; a two-dimensional structure (or a cured film) such as a film shape, a sheet shape, and a plate shape; and a one-dimensional structure such as linear (line shape), fibrous (fiber shape), and rod shape.

The method for producing the cured product is not particularly limited to a specific method. For example, the cured product may be produced by molding or casting (injecting) the curable composition into a predetermined mold, depending on the shape of the cured product; and then subjecting to a curing treatment (heating and/or light irradiation). The cured product having the two-dimensional structure may be produced by applying the curable composition to the base materials or substrates to form a film-shaped coating film (or thin film) and then by subjecting to the curing treatment. Examples of the base materials or substrates may include a metal such an aluminum; an inorganic material or ceramics such as titanium oxide, a glass and quartz; an organic material or plastic such as a cyclic olefinic resin and a polycarbonate resin; and a porous material such as wood.

Since the cured product of the present disclosure is formed with the di(meth)acrylate compound represented by the formula (1), the cured product has a high refractive index. Therefore, the cured product may have a refractive index nD at a temperature of 25° C. and a wavelength of 589 nm of about 1.6 to 1.8; a preferred range of the refractive index nD is 1.63 to 1.77, 1.65 to 1.75, 1.66 to 1.74, 1.67 to 1.73, 1.68 to 1.72, 1.685 to 1.715, 1.69 to 1.71, and 1.695 to 1.705 in a stepwise manner.

A refractive index of the curable composition before curing (refractive index of the curable composition at a temperature of 25° C. and a wavelength of 589 nm) nD may be about 1.59 to 1.8; and a preferred range of the refractive index of the curable composition is 1.6 to 1.77, 1.63 to 1.75, 1.65 to 1.72, 1.66 to 1.7, 1.665 to 1.695, 1.67 to 1.69, and 1.675 to 1.685 in a stepwise manner.

Further, the cured product has also a high heat resistance, and may have, for example, a 5% mass reduction temperature of about 200 to 500° C.; and a preferred range of the 5% mass reduction temperature is 300 to 450° C., 330 to 430° C., 340 to 420° C., 350 to 410° C., 360 to 400° C., 365 to 395° C., 370 to 390° C., and 375 to 385° C. in a stepwise manner.

The cured product has a high refractive index, and an unexpectedly low glass transition temperature Tg to exhibit a relatively flexible property (or toughness) regardless of high 5% mass reduction temperature. Therefore, the cured product may have, for example, a glass transition temperature Tg of about −30 to 100° C.; and a preferred range of the glass transition temperature is −10 to 70° C., 0 to 50° C., 5 to 40° C., 10 to 35° C., 15 to 30° C., and 20 to 25° C. in a stepwise manner.

The curable composition may have, for example, a viscosity at 25° C. of about 10 to 1000000 mPa-s, and the viscosity is preferably 30 to 100000 mPa s, and more preferably 50 to 60000 mPa-s.

Since the di(meth)acrylate compound represented by the formula (1) has an excellent solubility as mentioned above, the curable composition can have a viscosity adjusted with a solvent according to the applications and can form a highly uniform coating film or cured product. Therefore, the cured product having the two-dimensional structure such as film-shaped (cured film or hardened film) can be easily formed by a conventional coating method; and the cured film may have, for example, a thickness of about 50 nm to 300 μm, and for example about not more than 1 μm, preferably 80 to 200 nm, and more preferably 100 to 150 nm. Even a cured product of such a thin film can be efficiently or easily formed.

In this description and claims, the refractive index of the cured product or curable composition, the 5% mass reduction temperature, the glass transition temperature, the viscosity of the curable composition and the thickness of the cured product (cured film) can be measured according to the methods described in the following Examples.

### EXAMPLES

The following examples are intended to describe this disclosure in further detail and should by no means be interpreted as defining the scope of the disclosure. The detail of the raw materials and evaluation methods are shown below.

[Raw Materials]

DNPOA: 9,9-bis(3-acryloyloxypropyl)-2,7-di(2-naphthyl)fluorene, which was prepared in the following Example 1

BNEFA: 9,9-bis[6-(2-acryloyloxyethoxy)-2-naphthyl]fluorene, which was prepared in the following Comparative Example 1

BPEFA: 9,9-bis[4-(2-acryloyloxyethoxy)phenyl]fluorene, manufactured by Osaka Gas Chemicals Co., Ltd. (the following Comparative Example 2)

BPEF-9EOA: a diacrylate of a 9,9-bis[4-(2-hydroxyethoxy)phenyl]fluorene (or BPEF)-ethylene oxide (EO) adduct in which 9 mol on average of ethylene oxide is added to 1 mol of a 9,9-bis[4-(2-hydroxyethoxy)phenyl]fluorene; the adduct was prepared in the same manner as described in Reference Example 4 of Japanese Patent Application Laid-Open Publication No. 2013-53310.

POA: 2-phenoxyethyl acrylate, manufactured by Kyoeisha Chemical Co., Ltd., “LIGHT ACRYLATE PO-A”

OPPEOA: o-phenylphenoxyethyl acrylate, manufactured by Nippon Kayaku Co., Ltd.

[Evaluation Method]

(HPLC)

The HPLC purity [area] of the sample was measured using high performance (or high speed) liquid chromatography (HPLC) based on the following measuring equipment and conditions.

Equipment: manufactured by Hitachi High-Technologies Corporation, “L-2000”

Column: Imtakt Corporation, “Cadenza CL-C18 (3 μm) 3.0×250 mm”

Guard column: Imtakt Corporation, “GCCDOS”

Detector: L-2420 UV-VIS detector (D2 lump, 254 nm)

Mobile phase: acetonitrile/distilled water (volume ratio)=90/10 (manufactured by KANTO CHEMICAL CO., INC., LC grade)

Flow rate: 0.5 mL/min

(FD-MS)

Mass spectrometry (MS) was performed based on the following measuring equipment and conditions.

Equipment used: manufactured by JEOL Ltd., “JMS-T200GC”

Ionization: FD (Field desorption)

Emitter: carbon

Emitter current: 0 to 50 mA (25 mA/min)

(¹H-NMR)

The sample was dissolved in a heavy solvent (CDCl₃ or DMSO-d₆) containing tetramethylsilane as an internal standard substance, and the ¹H-NMR spectrum was measured using a nuclear magnetic resonance apparatus (manufactured by Bruker Corporation, “AVANCE III HD”).

(Melting Point)

The melting point was measured using a Differential Scanning Calorimeter (manufactured by SII NanoTechnology Inc., “EXSTAR DSC6200”) in an atmosphere of a nitrogen gas, at a measurement temperature of 30 to 220° C., and a heating rate (programming rate) of 10° C./min.

(5% Mass Reduction Temperature)

The temperature, at which the mass of the sample is reduced by 5% by mass was measured using a Thermogravimeter-Differential Thermal Analyzer (TG-DTA) (manufactured by SII NanoTechnology Inc., “TG/DTA6200”) in an atmosphere of a nitrogen gas, at a heating rate (programming rate) of 10° C./min. The sample (cured product) was prepared as follows.

That is, 3 parts by mass of Irgacure 184 (manufactured by BASF Japan Ltd.) as a photopolymerization initiator was added to 100 parts by mass of the total amount of the acrylate compound (polymerization component) shown in Table 1 or 3 weighed in a brown bottle, and the mixture was heated to 120° C. for dissolution to obtain a curable composition. An UV irradiation (500 mJ/cm²) was repeated 4 times on the obtained curable composition to prepare the cured product.

(Glass Transition Temperature Tg)

The glass transition temperature Tg was measured using a Differential Scanning Calorimeter (manufactured by SII NanoTechnology Inc., “EXSTAR 6000 DSC6220 ASD-2”) in an atmosphere of a nitrogen gas at a heating rate (programming rate) of 10° C./min. The sample (cured product) was prepared in the same manner as the measurement sample (cured product) of 5% mass reduction temperature.

(Refractive Index Before Curing nD)

The refractive index before curing was measured at a temperature of 25° C. and a wavelength of 589 nm (D line). As the refractive index meter, Multi-wavelength Abbe Refractometer (manufactured by ATAGO CO., LTD., “DR-M2 (circulatintg constant-temperature bath 60-C₃)”) was used in Example 1 and Comparative examples 1 to 4, and Digital Refractometer (manufactured by ATAGO CO., LTD., “RX-7000i”) was used in Examples 2 to 10.

The refractive index of DNFDP-m obtained in Example 1 was calculated by extrapolating the concentration to 100% by mass on the calibration curve (approximate straight line) which prepared by dissolving the sample in chloroform to prepare a solution with concentrations 7.673 and 16.8% by mass each, and measuring the refractive index of the obtained solutions.

The refractive index of DNPOA obtained in Example 1 was calculated by extrapolating the concentration to 100% by mass on the calibration curve (approximate straight line) which prepared by dissolving the sample in toluene to prepare a solution with concentrations 25.0, and 48.7% by mass each, and measuring the refractive index of the obtained solutions.

The refractive index of BNEFA obtained in Comparative Example 1 was also calculated by extrapolating the concentration to 100% by mass on the calibration curve (approximate straight line by least squares method) which prepared by measuring the refractive index of plural toluene solutions with different concentrations.

(Refractive Index nD of Cured Product (Cured Film) and Film Thickness Thereof)

The refractive index of the cured product was measured at a temperature of 25° C. and a wavelength of 589 nm (D line) for the cured film obtained by photo-curing the sample. The preparation method of the sample (cured film) and the measurement equipment are shown below.

In Example 1; 3 parts by mass of Irgacure 184 (manufactured by BASF Japan Ltd.) as a photopolymerization initiator was added to 100 parts by mass of DNPOA weighed in a brown bottle, and the mixture was diluted with toluene. This diluted solution (or curable composition) was dropped onto the surface of a silicon wafer of about 3×3 cm and spin-coated (1000 rpm, 30 sec) to form a thin coating layer which was then irradiated with UV (500 mJ/cm²) to prepare a cured film. The thickness and refractive index nD of the obtained cured film were measured using the high speed spectroscopic ellipsometer (manufactured by J. A. Woollam Co., Inc., “M-2000”).

In Comparative Examples 2 to 4 and Examples 5 and 8 to 10; 3 parts by mass of Irgacure 184 (manufactured by BASF Japan Ltd.) as a photopolymerization initiator was added to 100 parts by mass of the total amount of the acrylate compound shown in Table 1 or 3 (polymerization component) weighed in a brown bottle, and the resultant was heated to form a molten mixture. The obtained curable composition was applied onto a TAC (cellulose acetate) film to form a film (layer) having a thickness of 200 to 400 μm by using an applicator, and the obtained coating film (layer) was irradiated with UV (500 mJ/cm²) once to prepare a cured film.

The refractive index nD of the obtained cured film was measured by using Multi-wavelength Abbe Refractometer (manufactured by ATAGO CO., LTD., “DR-M2 (circulating constant-temperature bath 60-C₃)”), and the thickness of the obtained cured film was measured by using the equipment “MDQ-30” manufactured by Mitutoyo Corporation.

(Viscosity)

The viscosity (melting viscosity) at 150° C. of DNPOA obtained in Example 1 was measured under a rotation speed of 900 rpm with selecting an optional rotor (cone 6) by using CAP2000+ Viscometer (manufactured by Brookfield) depending on the measured viscosity.

In Examples 2 to 10 and Comparative Examples 2 to 4; the viscosity at 25° C. was measured under a rotation speed of 0.5 to 20 rpm with selecting an optional rotor (01:1°34′×R24, or 07:3°×R7.7) by using TV-22 Viscometer (cone-plate type; which manufactured by TOKI SANGYO CO., LTD, “TVE-22L”) depending on the measured viscosity.

(Solubility)

The sample 0.3 g was weighed in a sample bottle, the solvent was added by 0.7 g (condition 1: solid content of 30% by mass) or 1.0 g (condition 2: solid content of 23% by mass), and each of the obtained mixed solutions was stirred at a temperature of 50° C. for 30 minutes by using a bioshaker (manufactured by TAITEC CORPORATION, “BR-43FH”). Each of the mixed solutions after stirring was evaluated according to the following criteria. The used solvents were methyl ethyl ketone (MEK), methyl isobuthyl ketone (MIBK), ethyl acetate, propylene glycol monomethyl ether acetate (PGMEA), toluene, dimethylformamide (DMF), or cyclohexanone.

A: Soluble

B: Some soluble, but turbid

C: Insoluble

(Curability)

The curability was evaluated according to the following criteria from the tough feel of the surface of the cured product (cured film) which was prepared for specifying the refractive index nD of the cured product.

A: There is no tackiness (adhesiveness) on the surface of the cured product.

B: There is a tackiness (adhesiveness) on the surface of the cured product.

### Example 1

(Preparation of DBrFDP-m)

9,9-bis(2-methoxycarbonylethyl)-2,7-dibromofluorene (DBrFDP-m) was synthesized in the same manner as described in Example 1 of Japanese Patent Application Laid-Open Publication No. 2005-89422 except that methyl acrylate [37.9 g (0.44 mol)] was used instead of t-butyl acrylate, and 2,7-dibromo-9H-fluorene [54.7 g (0.17 mol)] was used instead of fluorene.

(Preparation of DNFDP-m)

After charging 192.3 g (0.39 mol) of DBrFDP-m, 200 g (1.2 mol) of 2-naphthylboronic acid, 4.3 L of dimethoxyethane, and 1 L of 2M sodium carbonate aqueous solution into a reactor, 22.4 g (19.4 mmol) of tetrakis(triphenylphosphine)palladium(0) [or Pd(PPh₃)₄] was added to the mixture under an nitrogen stream, and the resultant mixture was heated under reflux at an internal temperature of 71 to 78° C. for 5 hours for reaction. After cooling to a room temperature, 2.0 L of toluene and 500 mL of ion-exchanged water were added to the reaction mixture, and the mixture was subjected to extraction (liquid-liquid extraction) 5 times with solvents (2.0 L of toluene and 500 mL of ion-exchanged water) for washing. The color of the organic layer changed from dark orange to brown. The insoluble material was filtered off and the filtrate was concentrated to obtain 305 g of brown crude crystals. The crude crystals were dissolved; under heating, in a mixed solution of 1.5 kg of ethyl acetate and 300 g of isopropyl alcohol (IPA), and then the mixture was cooled to not higher than 10° C. with ice water, and stirred for 1 hour to precipitate crystals. The precipitated crystals were filtered, and then were dried under reduced pressure to obtain 130 g of taupe crystals. The taupe crystals were purified by column chromatography (Silica gel carrier, Developing solvent: chloroform:ethyl acetate (volume ratio)=4:1), and then were recrystallized from methanol and dried under reduced pressure to obtain 116 g of 9,9-bis(2-methoxycarbonylethyl)-2,7-di(2-naphthyl)fluorene (DNFDP-m) represented by the following formula (White crystals, Yield 54.9 A, and HPLC purity 99.4 area %). The results of ¹H-NMR and FD-MS are shown below.

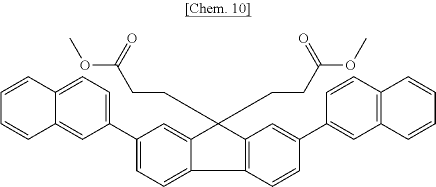 [SMILES: COC(=O)CCCC1(CCCC(=O)OC)c2cc(-c3ccc4ccccc4c3)ccc2-c2ccc(-c3ccc4ccccc4c3)cc21]

¹H-NMR (CDCl₃, 300 MHz): δ (ppm) 1.7 (t, 4H), 2.6 (t, 4H), 3.4 (s, 6H), 7.5 (m, 4H), 7.7-8.0 (m, 14H), 8.1 (s, 2H)

FD-MS: m/z 590 (M+)

The obtained DNFDP-m had the refractive index nD of 1.845, the melting point of 191° C., and the 5% mass reduction temperature of 390° C.

### Preparation of 2,7-dinaphthylfluorene-9,9-dipropanol

After charging 201 g (0.340 mol) of DNFDP-m into a 3 L of reactor equipped with a stirrer, a dropping funnel, and a three-way cock, and replacing the inner atmosphere of the reactor with nitrogen, 1.7 L of tetrahydrofuran (THF) was poured into the reactor for dissolution, and the mixture was cooled with water. Under water cooling, 52.4 g (1.38 mol) of sodium borohydride was dividedly added to the mixture over 5 minutes, then 174 mL (1.38 mol) of boron trifluoride-diethyl ether complex was added dropwise to the reactant over 1 hour, and the mixture was stirred at room temperature for 22 hours. The progress of the reaction was confirmed by HPLC. After the reaction, THF was distilled off from the reaction mixture under heating and reduced pressure (Outside temperature of 45° C., Diaphragm pump), then 2.5 L of dichloromethane was added to the condensed mixture, the mixture was stirred for 1 hour for dissolution, washed 3 times with 1.5 L of purified water, and dried over sodium sulfate. After separating sodium sulfate by filtration, the filtrate was concentrated for solidification under heating and reduced pressure (outside temperature of 60° C., oil rotary pump) to obtain 176 g of 2,7-dinaphthylfluorene-9,9-dipropanol [or 9,9-bis(3-hydroxypropyl)-2,7-di(2-naphthyl)fluorene] represented by the following formula as a white solid in a yield of 96.9%. The result of ¹H-NMR is shown below.

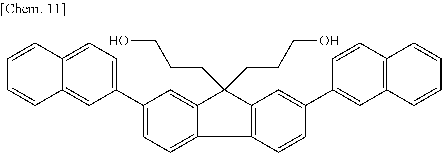 [SMILES: OCCCC1(CCCO)c2cc(-c3ccc4ccccc4c3)ccc2-c2ccc(-c3ccc4ccccc4c3)cc21]

¹H-NMR (DMSO-d₆, 300 MHz) δ (ppm) 0.9 (m, 4H), 2.2 (m, 4H), 3.2 (t, 4H), 4.2 (t, 2H), 7.5-8.4 (m, 20H)

(Preparation of DNPOA)

After charging 30.0 g (0.06 mol) of 2,7-dinaphthylfluorene-9,9-dipropanol, 10.5 g (0.15 mol) of acrylic acid, 55 g of toluene, and 0.12 g (1.0 mmol) of 2-methoxyphenol into a 500 mL three neck flask with Dean-Stark, the inner atmosphere of the system was replaced with nitrogen, the temperature was raised to 95° C. After the mixture was made homogeneous, 1.33 g (7.0 mmol) of p-toluenesulfonic acid monohydrate was added to the reaction system, the inner atmosphere was replaced with nitrogen again, and the by-product water was removed with refluxing for 4 hours. The reaction temperature was 110 to 115° C.

The obtained mixture was washed with 195 g of toluene and 20 g of 20% by mass saline (internal temperature of 60 to 70° C.), and then neutralized with 20 g of 10% caustic soda (10% by mass sodium hydroxide aqueous solution) and 20 g of 20% by mass saline (internal temperature of 60 to 70° C.), to confirm that the aqueous layer had a pH of 10 or higher. After 500 ppm by mass of 2-methoxyphenol was added to total organic layer, the solution was made uniform (homogeneous), and the solution was washed twice with 20 g of 20% by mass saline and twice with 20 g of ion-exchanged water (internal temperature of 60 to 70° C.) to confirm that the aqueous layer had a pH of 7. Then, 6 g of activated carbon (manufactured by Mizusawa Industrial Chemicals, Ltd., “FP-6”) was added to the organic layer, and the mixture was stirred at room temperature for 1 hour, filtered through cerite, concentrated, and dried under reduced pressure at 100° C. overnight to obtain 2,7-dinaphthylfluorene-9,9-dipropyldiacrylate [or 9,9-bis(3-acryloyloxypropyl)-2,7-di(2-naphthyl)fluorene](DNPOA) represented by the following formula as a pale yellow solid (HPLC purity 93.2%). The result of ¹H-NMR is shown below. Further, each evaluation was performed for the obtained DNPOA.

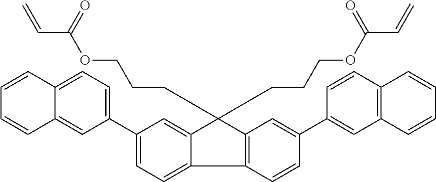 [SMILES: C=CC(=O)OCCCCC1(CCCCOC(=O)C=C)c2cc(-c3ccc4ccccc4c3)ccc2-c2ccc(-c3ccc4ccccc4c3)cc21]

¹H-NMR (CDCl₃, 300 MHz): δ (ppm) 1.1 (m, 4H), 2.3 (m, 4H), 3.9 (t, 4H), 5.7 (dd, 2H), 6.0 (dd, 2H), 6.3 (dd, 2H), 7.5-8.1 (m, 20H)

### Comparative Example 1

BNEFA represented by the following formula was prepared according to Synthesis Example 1 of Japanese Patent Application Laid-Open Publication No. 2018-59059 (Patent Document 2), and subjected to each evaluation.

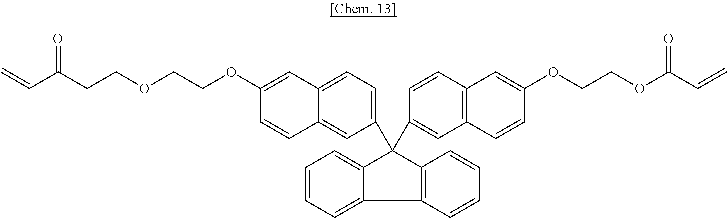 [SMILES: C=CC(=O)CCOCCOc1ccc2cc(C3(c4ccc5cc(OCCOC(=O)C=C)ccc5c4)c4ccccc4-c4ccccc43)ccc2c1]

### Comparative Example 2

BPEFA represented by the following formula (manufactured by Osaka Gas Chemicals Co., Ltd.) was used and subjected to each evaluation.

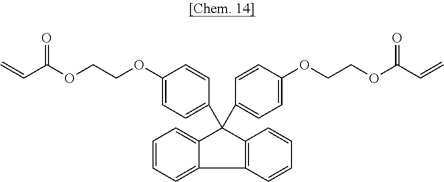 [SMILES: C=CC(=O)OCCOc1ccc(C2(c3ccc(OCCOC(=O)C=C)cc3)c3ccccc3-c3ccccc32)cc1]

The evaluated results are shown in Table 1.

[Table 1]

TABLE 1

| | Before curing | | | | Cured product | | | |
|---|---|---|---|---|---|---|---|---|
| | | | | Melting | | Film | | 5% mass |
| | Appearance | Viscosity | nD | point | nD | thickness | Tg | reduction |
| Compound | (25° C.) | [mPa · s] | (25° C.) | [° C.] | (25° C.) | [nm] | [° C.] | temperature[° C.] |
| Example 1 | Pale yellow | 258 | 1.682 | 120 | 1.701 | 120 | 23 | 382 |
| DNPOA | solid | (150° C.) | | | | | | |
| Comparative | White | — | 1.652 | — | — | — | — | — |
| Example 1 | powder | | | | | | | |
| BNEFA | | | | | | | | |
| Comparative | Viscous | >352000 | 1.616 | — | 1.626 | — | 211 | — |
| Example 2 | body | (25° C.) | | | | | | |
| BPEFA | | | | | | | | |

As apparent from Table 1, DNPOA obtained in Example 1 had a remarkably high refractive index nD. As shown by the conventional acrylate compound of Comparative examples 1 to 2, the refractive index tends to improve or increase with the increase of the aromatic ring skeleton (benzene ring skeleton) in the molecular structure. In contrast, unexpectedly, the refractive index of DNPOA in Example 1 was improved by 0.03 with respect to BNEFA in Comparative Example 1, despite the only fact that DNPOA and BNEFA have the same number of aromatic ring skeletons, and naphthalene rings are bonded at different positions of fluorene skeleton. Such an effect means a remarkable effect, since it is usually evaluated to have a superiority, even if the refractive index increases by about 0.01.

The cured product of DNPOA which was obtained in Example 1 also had a high 5% mass reduction temperature. Thus, in spite of having a high refractive index and heat resistance, unexpectedly, DNPOA had a lower glass transition temperature.

Furthermore, Table 2 shows the evaluation results of solubility in various solvents for Example 1 (DNPOA) and Comparative Example 1 (BNEFA).

[Table 2]

TABLE 2

| | |
|---|---|
| | Comparative |
| Example 1 | Example 1 |
| DNPOA | BNEFA |

| Solvent | 30% | 23% | 30% | 23% |
|---|---|---|---|---|
| MEK | A | — | C | — |
| MIBK | A | — | C | B |
| Ethyl acetate | A | — | C | B |
| PGMEA | B | A | A | — |
| Toluene | A | — | C | — |
| DMF | A | — | A | — |
| Cyclohexanone | A | — | B | B |

As apparent from Table 2, Comparative Example 1 (BNEFA) shows a high frequency of evaluation criteria C and B, and has a lower solubility. These results mean that the aromatic ring skeleton in the molecular structure tends to improve the refractive index and contrarily decrease the solubility, that is, it is difficult to be compatible high refractive index with high solubility (handleability). On the other hand, DNPOA obtained in Example 1 has high solubility in various solvents even though the number of aromatic ring skeletons is the same as the number of aromatic ring skeletons of BNEFA, and had an excellent in balance with refractive index and solubility.

### Examples 2 to 10, and Comparative Examples 3 to 4

The acrylate compounds were mixed in the mass ratios shown in Table 3 below, to prepare the curable composition containing the obtained mixture for subjecting each evaluation. The numbers in parentheses in the column of acrylate compound in Table 3 mean parts by mass.

TABLE 3

| | Curable composition | | | | Cured product | | | |
|---|---|---|---|---|---|---|---|---|
| | | | | | | | 5% mass | Film |
| | Acrylate | | Viscosity(25° C.) | nD | nD | | reduction | thickness |
| | compound | | [mPa · s] | (25° C.) | (25° C.) | Curability | temperature[° C.] | [μm] |
| Comparative | BNEFA | POA | 27000 | 1.609 | 1.640 | A | — | — |
| Example 3 | (70) | (30) | | | | | | |
| Example 2 | DNPOA | POA | 41420 | 1.643 | — | — | — | — |
| | (70) | (30) | | | | | | |
| Example 3 | DNPOA | POA | 645 | 1.608 | — | — | — | — |
| | (50) | (50) | | | | | | |
| Example 4 | DNPOA | POA | 63 | 1.571 | — | — | — | — |
| | (30) | (70) | | | | | | |
| Comparative | BNEFA | OPPEOA | >352000 | 1.633 | 1.649 | A | — | — |
| Example 4 | (70) | (30) | | | | | | |
| Example 5 | DNPOA | OPPEOA | >352000 | 1.661 | 1.682 | A | 224.9 | 220 |
| | (70) | (30) | | | | | | |
| Example 6 | DNPOA | OPPEOA | 33140 | 1.637 | — | — | — | — |
| | (50) | (50) | | | | | | |
| Example 7 | DNPOA | OPPEOA | 2006 | 1.614 | — | — | — | — |
| | (30) | (70) | | | | | | |
| Example 8 | DNPOA | BPEF-9EOA | >352000 | 1.651 | 1.680 | A | 354.7 | 150 |
| | (70) | (30) | | | | | | |
| Example 9 | DNPOA | BPEF-9EOA | 58900 | 1.618 | 1.641 | A | 329.1 | 100 |
| | (50) | (50) | | | | | | |
| Example 10 | DNPOA | BPEF-9EOA | 45500 | 1.597 | 1.616 | A | 345.1 | 100 |
| | (30) | (70) | | | | | | |

As apparent from Table 3, the curable compositions of Examples 2 to 10 containing DNPOA of Example 1 also had a significantly high refractive index nD. On the other hand, the curable compositions of Comparative Examples 3 to 4 containing BNEFA of Comparative Example 1 instead of DNPOA had a lower refractive index in comparison of the curable composition corresponding Examples 2 and 5, respectively. Further, in Examples 3 and 6 having a refractive index close to those of Comparative Examples 3 to 4, the viscosity was remarkably low and the handleability was excellent. Therefore, according to Examples, both high refractive index and low viscosity can be achieved in a well-balanced manner. [(0298] Further, the cured products of Examples also had a high 5, mass reduction temperature. Therefore, the cured products of Examples had a high refractive index and a heat resistance.

### INDUSTRIAL APPLICABILITY

The di(meth)acrylate compound (or curable composition or cured product thereof) of the present disclosure has excellent optical properties such as high refractive index and high heat resistance. Therefore, the di(meth)acrylate compound (or curable composition or cured product thereof) can be used for various applications, for example, a coating agent or a coating film or layer, specifically a paint, an ink, or a protective film or layer for an electronic device and a liquid crystal member (component); an adhesive, or a pressure-sensitive adhesive; a resin filler; an electrical/electronic material or an electrical/electronic component (electrical/electronic equipment), specifically an electrical/electronic material or an electrical/electronic component such as an antistatic agent, a carrier transport agent, a light emitter, an organic photoconductor, a thermal recording material, a photochromic material, a hologram recording material, a charging (static) tray, a conductive sheet, an optical disk, an inkjet printer, a digital paper, a color filter, an organic EL element, an organic semiconductor laser, a dye-sensitized solar cell, a sensor, or an EMI shield film; a machine material or machine part (equipment), specifically a machine material or machine part such as an automotive material or part, an aerospace-related material or part, and slide member.

In particular, the di(meth)acrylate compound (or curable composition or cured product thereof) of the present disclosure can be effectively used for an optical member (optical element) or an optical material, for example, an optical adhesive (sealing agent) or an optical pressure-sensitive adhesive, such as OCR (Optical Clear Resin), OCA (Optical Clear Adhesive) tape or film; an optical film (optical sheet); an optical lens; a prism; a hologram; and an optical fiber.

Examples of the optical film may include a polarizing film, a polarizing element constituting the polarizing film and a polarizing plate protective film, a retardation film (phase contrast film), an oriented film (alignment film), a wide view (compensation) film, a diffuser plate (film), a prism sheet, a light guide plate, a brightness enhancement film, a near infrared absorbing film, a reflective film, an anti-reflective (AR) film, a low reflective (LR) film, an antiglare (AG) film, a transparent conductive (ITO) film, an anisotropic conductive film (ACF), an electromagnetic wave shield (EMI) film, a film for electrode substrate, a film for color filter substrate, a barrier film, a color filter layer, a black matrix layer, and an adhesive layer between optical films or release layer. The optical film may be an optical film for a display such as a liquid crystal display (LCD), an organic EL display (OLED), a plasma display (PDP), a field emission display (FED), and an electronic paper.

Examples of the optical lens may include a lens for glasses, a contact lens, a lens for a camera, a VTR zoom lens, a pickup lens, a Fresnel lens, a solar condenser lens, an objective lens, a rod lens array and other lens.

1. A compound represented by the following formula (1): 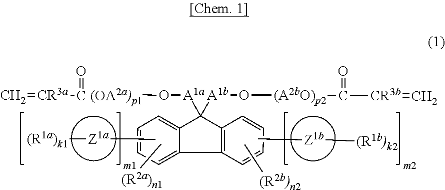
wherein Z^{1a} and Z^{1b} independently represent an arene ring,
R^{1a} and R^{1b} independently represent a substituent, k1 and k2 independently denote an integer of not less than 0,
m1 and m2 independently denote an integer of 0 to 4, and at least one of m1 and m2 denotes 1 or more,
R^{2a} and R^{2b} independently represent a substituent, n1 and n2 independently denote an integer of 0 to 4,
m1+n1 and m2+n2 each denote 4 or less,
A^{1a} and A^{1b} independently represent a straight- or branched-chain alkylene group,
A^{2a} and A^{2b} independently represent a straight- or branched-chain alkylene group, p1 and p2 independently denote an integer of not less than 0, and
R^{3a} and R^{3b} independently represent a hydrogen atom or a methyl group.

2. The compound according to claim 1, wherein, in the formula (1), Z^{1a} and Z^{1b} each represent a C₆₋₁₂arene ring,
m1 and m2 each denote an integer of 1 to 2,
A^{1a} and A^{1b} each represent a straight- or branched-chain C₁₋₄alkylene group,
A^{2a} and A^{2b} each represent a straight- or branched-chain C₂₋₄alkylene group, and p1 and p2 each denote an integer of 0 to 10.

3. The compound according to claim 1, wherein, in the formula (1), Z^{1a} and Z^{1b} each represent a benzene ring or a naphthalene ring,
m1 and m2 each denote 1,
A^{1a} and A^{1b} each represent a straight- or branched-chain C₁₋₄alkylene group, and
p1 and p2 each denote 0.

4. The compound according to claim 1, which has a refractive index of 1.65 to 1.75 at a wavelength of 589 nm and a temperature of 20° C.

5. A process for producing a compound recited in claim 1, which comprises allowing a compound represented by the following formula (2): 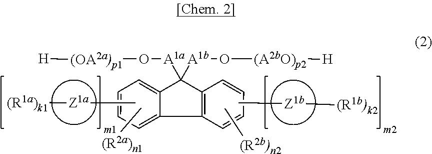
wherein Z^{1a} and Z^{1b}, R^{1a} and R^{1b}, k1 and k2, m1 and m2, R^{2a} and R^{2b}, n1 and n2, m1+n1 and m2+n2, A^{1a} and A^{1b}, A^{2a} and A^{2b}, and p1 and p2 each have the same meanings as defined in the formula (1),
to react with compounds represented by the following formulae (3a) and (3b): 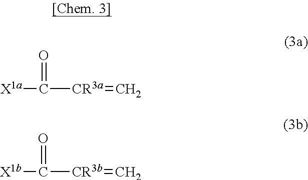
wherein X^{1a} and X^{1b} independently represent a hydroxy group, an alkoxy group, or a halogen atom, and R^{3a} and R^{3b} each have the same meanings as defined in the formula (1).

6. A curable composition containing a compound recited in claim 1.

7. The curable composition according to claim 6, which further contains a compound represented by the following formula (7): 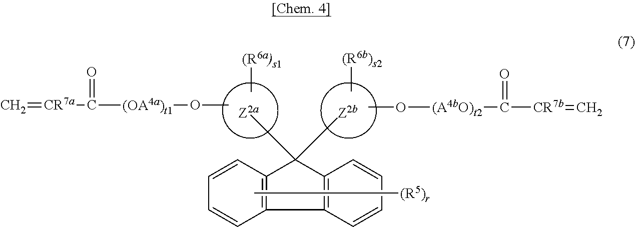
wherein Z^{2a} and Z^{2b} independently represent an arene ring,
R⁵ represents a substituent, r denotes an integer of 0 to 8,
R^{6a} and R^{6b} independently represent a substituent, s1 and s2 independently denote an integer of not less than 0,
A^{4a} and A^{4b} independently represent a straight- or branched-chain alkylene group, t1 and t2 independently denote an integer of not less than 0, and
R^{7a} and R^{7b} independently represent a hydrogen atom or a methyl group.

8. The curable composition according to claim 7, wherein, in the formula (7), Z²a and Z^{2b} each represent a C₆₋₁₂arene ring,
R^{6a} and R^{6b} each represent a hydrocarbon group, s1 and s2 each denote an integer of 0 to 2,
A⁴a and A^{4b} each represent a straight- or branched-chain C₂₋₄alkylene group, t1 and t2 each denote an integer of 0 to 10; and
a mass ratio of the compound represented by the formula (1) relative to the compound represented by the formula (7) is 10/90 to 90/10 in terms of the former/the latter.

9. The curable composition according to claim 6, which further contains a compound represented by the following formula (8): 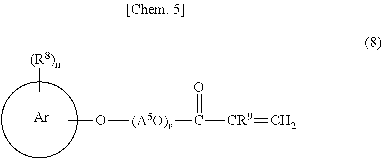
wherein Ar represents an arene ring,
R⁸ represents a substituent, u denotes an integer of not less than 0,
A⁵ represents a straight- or branched-chain alkylene group, v denotes an integer of not less than 0, and
R⁹ represents a hydrogen atom or a methyl group.

10. The curable composition according to claim 9, wherein, in the formula (8), Ar represents a C₆₋₁₂arene ring,
R⁸ represents a hydrocarbon group, u denotes an integer of 0 to 2,
A⁵ represents a straight- or branched-chain C₂₋₄alkylene group, v denotes an integer of 1 to 4; and
a mass ratio of the compound represented by the formula (1) relative to the compound represented by the formula (8) is 10/90 to 95/5 in terms of the former/the latter.

11. A cured product in which a curable composition recited in claim 6 has been cured.

12. The cured product according to claim 11, which has:
a refractive index of 1.65 to 1.75 at a wavelength of 589 nm and a temperature of 20° C.,
a glass transition temperature of 0 to 50° C., and
a 5% mass reduction temperature of 330 to 430° C.

13. An optical member containing a cured product recited in claim 11.
